(19) United States
(12) Patent Application Publication
(10) Pub. No.: US 2004/0198705 A1
(43) Pub. Date: Oct. 7, 2004

(54) Modulation of steroid hormone uptake

(76) Inventors: Thomas Willnow, Berlin (DE); Anders Nykjaer, Risskov (DK)

Correspondence Address: Ronald W Citkowski, Gifford Krass Groh Sprinkle Anderson & Citkowski, Suite 400, 280 North Old Woodward Avenue, Birmingham, MI, 48009, US

(21) Appl. No.: 10/479,875

(22) PCT Filed: Jun. 4, 2002
(86) PCT No.: PCT/DK02/00379

Related U.S. Application Data
(60) Provisional application No. 60/296,489, filed on Jun. 8, 2001.

(30) Foreign Application Priority Data
Jun. 7, 2001 (DK) PA 2002-00887

(51) Int. Cl. G01N 33/53; A61K 31/56

(52) U.S. Cl. 514/169; 435/7.1

(57) ABSTRACT

The present invention discloses that cells, such as mammalian cells actively can take up steroid hormones/steroid hormone binding protein complexes via steroid hormone binding protein receptors and/or co-receptors. Furthermore, the invention relates to methods for screening for compounds, which alters uptake of such complexes, compounds identified by said methods as well as methods for treatment using said compounds and uses of said compounds for the preparation of a medicament.

### BACKGROUND OF THE INVENTION

Many developmental processes in a wide variety of species are regulated by steroid hormones, all of which are synthesised from cholesterol. Being relatively small hydrophobic molecules, they are thought to be able to cross the plasma membrane by simple diffusion. Once inside the target cell, each type of steroid hormone binds tightly but reversibly to specific nuclear receptors that act as transcriptional regulators.

In the circulation steroid hormones are transported by specific plasma carrier proteins. These carriers include the sex hormone-binding globulin (SHBG), the corticoidsteroid-binding globulin (CBG) and the vitamin D-binding protein (DBP). SHBG specifically binds androgens and estrogens. Due to the high affinity between carrier and ligand and because of an excess of carrier, more than 95% of circulating steroid hormones are found in complex with their respective carriers.

According to one theory, the role of the carriers is to keep bound steroids in a biological inactive state and to regulate the plasma concentration of free hormone, which can enter the cells by passive diffusion. This concept of steroid uptake requires unspecific entry of steroid hormones into all cells of an organism in order to find their intracellular receptors. Furthermore, the amount of steroids that can be taken up depends on the systemic concentration of the free hormones and may not be increased locally in steroid responsive-tissues.

DBP is the principal carrier of vitamin D metabolites in plasma and it is estimated that only 0.003% vitamin D₃ metabolites are found in the unbound form. Epithelial cells of the renal proximal tubuli are responsible for the conversion of 25-OH vitamin D₃ to its active form. However, the amount of 25-OH vitamin D₃ that is taken up and converted by the kidney considerably exceeds the amount of precursor that can be taken up by passive diffusion only. It has been demonstrated that megalin, a member of the low density lipoprotein (LDL) receptor family is a receptor for DBP and internalises 25-OH vitamin D₃/DBP complexes into the tubular epithelium. Lack of the receptor in megalin knock-out mice results in the inability of renal epithelial cells to take up 25-OH vitamin D₃ and hence to produce the active hormone. As a consequence, receptor deficient animals exhibit vitamin D3 deficiency (Nykjaer et al, 1999).

The LDL receptor is the founding member of a family of receptors that all share the same structural motifs and furthermore include LDL receptor-related protein (LRP), megalin, the very-low-density lipoprotein (VLDL) receptor and the apolipoprotein E receptor-2 (ApoER2), LRP-1 B, MEGF-7, chicken VTG receptor, Drosophila Y1 protein, gp 330/megalin and C. elegans gp330/megalin-related protein. The modular structure of the extracellular domains of the individual receptors is very similar and has been remarkably conserved throughout evolution. Each receptor comprises a number of complement type repeat clusters, for example does the LRP receptor contain 4 such clusters. The number of complement type repeats within each cluster varies from 2 to 11. Family members have been identified in flies as well as in the nematode C. elegans. In contrast to the extracellular domains, the cytoplamic tails of the different receptors share very little sequence similarity, with the exception of a short amino-acid motif characterised by the consensus sequence Asn-Pro-Xaa-Tyr, where Xaa can be any amino acid. This motif has been shown to mediate clustering of the LDL receptor in coated pits before endocytosis.

The LDL receptor is involved in the uptake of low density lipoproteins (LDL) from the blood. After binding of LDL, LDL receptors cluster on coated pits in the plasma membrane. This step is followed by the formation and internalisation of endocytic vesicles, hydrolysis of the endocytosed lipoproteins in lysosomes and release of the lipids into the cytoplasm.

LRP is involved in the uptake of chylomicron remnants, the carriers of dietary lipids, into hepatocytes. However, LRP furthermore acts as the receptor for the abundant plasma protease inhibitor α2-macroglobulin, as well as for a number of other protease inhibitors, proteases, lipases and other functionally diverse macromolecules.

Megalin is expressed abundantly on the apical surface of the epithelial cells of proximal tubules in the kidney and the major ligands of Megalin appears to be DBP and retinol-binding protein (RBP), which is a carrier for vitamin A.

### SUMMARY OF THE INVENTION

A number of steroid-responsive cells such as for example male germ cells and cells of prostate and endometrium and estrogen-dependent breast cancer cells have binding sites for various carrier proteins on their surfaces. For example SHBG binds to specific receptors in endometrium, prostate cells and breast cancer cells, and is presumably taken up by these cells via receptor mediated endocytosis (Porto et al., 1995).

Interestingly, the present invention discloses that cells, such as for example cells of epididymis and prostate actively take up steroid hormones/steroid hormone binding protein complexes, for example testosterone/SHBG complexes or complexes comprising estrogen, progesterone and/or corticoids by receptor mediated endocytosis and that the receptors involved in binding and internalising these complexes belong to the LDL receptor family. Furthermore, the present invention discloses that in some cases the presence of a co-receptor is required for the uptake of steroid hormone binding proteins into cells. These observations provide a unique possibility to specifically regulate the uptake of steroid hormones into steroid hormone responsive cells including cells that metabolise the steroid hormones.

Accordingly, it is a first aspect of the present invention to provide an in vitro method for screening for a compound which alters uptake of steroid hormones into cells presenting a steroid hormone binding protein receptor, said method comprising

a) providing an assay for measuring binding to or uptake by the steroid hormone binding protein receptor of steroid hormones bound to or complexed with a steroid hormone binding protein,

b) adding the compound to be tested to the assay, and

c) determining the amount of steroid hormones bound to or complexed with a steroid hormone binding protein which is bound to or taken up by the steroid hormone binding protein receptor,

d) comparing the amount determined in step c) with an amount measured in the absence of the compound to be tested,

e) wherein a difference in the two amounts identifies a compound which alters the binding or uptake of steroid hormones bound to or complexed with steroid hormone binding proteins.

It is a second objective of the present invention to provide a method of screening for a compound which alters uptake of steroid hormones into cells presenting a steroid hormone binding protein receptor, said method comprising

a) administering said compound to a mammal naturally expressing the receptor,

b) measuring expression of receptor mRNA or protein in tissues of the mammal or measuring receptor-mediated binding or transport of steroid hormones bound to or complexed with steroid hormone binding proteins or measuring uptake of steroid hormones or steroid hormone/steroid hormone binding protein complexes into cells of said mammal,

c) comparing the measurement of step b) with a measurement measured in the absence of the compound to be tested,

d) wherein a difference in the two measurements identifies a compound which alters the uptake of steroid hormones bound to or complexed with steroid hormone binding proteins.

Furthermore, it is an objective of the present invention to provide compounds identified by one of the above mentioned methods which alters the uptake of steroid hormone into cells presenting a steroid hormone binding protein receptor.

It is a further objective of the present invention to provide a method for determining the effect of a compound on uptake of steroid hormones into cells presenting a steroid hormone binding protein receptor, said method comprising the steps of

a) administering said compound to a mammal naturally expressing the receptor,

b) measuring expression of receptor mRNA or protein in tissues of the mammal or measuring receptor-mediated binding or transport of steroid hormones bound to or complexed with steroid hormone binding proteins or measuring uptake of steroid hormones or steroid hormone/steroid hormone binding protein complexes into cells of said mammal,

c) comparing the measurement of step b) with a measurement measured in the absence of the compound to be tested,

d) wherein the difference in the two measurements identifies the effect of said compound on the uptake of steroid hormones into cells presenting a steroid hormone binding protein receptor.

The cells of certain tumours are dependent on steroid hormones for growth. Hence, it is desirable to inhibit uptake of steroid hormones by such cells. In particular, this is the case for cells of prostate cancer and breast cancer.

Accordingly, it is a still further objective of the present invention to provide methods of treating prostate cancer comprising administering to an individual having prostate cancer a sufficient amount of a compound which alters the uptake of steroid hormone into cells presenting a steroid hormone binding protein receptor. It is also an objective to provide methods of treating breast cancer comprising administering to an individual having breast cancer a sufficient amount of a compound which alters the uptake of steroid hormone into cells presenting a steroid hormone binding protein receptor.

Additionally, it is an objective of the present invention to provide a method of preventing pregnancy comprising administering to an individual in need thereof a sufficient amount of a compound which alters the uptake of steroid hormone into cells presenting a steroid hormone binding protein receptor.

The present invention furthermore provides pharmaceutical compositions comprising a compound, which alters the uptake of steroid hormone into cells presenting a steroid hormone binding protein receptor as well as uses of said compound for the preparation of a pharmaceutical composition for the treatment of a condition selected from the group consisting of prostate cancer and breast cancer.

Models of receptor-mediated endocytosis of steroid hormone/steroid hormone binding protein complexes according to the present invention are depicted in FIGS. 4 and 5. In the circulation, steroid hormones are transported in a complex with specific steroid hormone binding proteins (carrier) such as sex hormone-binding globulin (SHBG), corticosteroid-binding globulin (CBG), Clara cell secretory protein (CCSP), apolipoprotein D (apoD) or 24p3. Target cells take up complexed steroid hormones via endocytic receptors recognising the steroid hormone binding protein (FIG. 4). Alternatively, a steroid hormone binding protein co-receptor mediates the binding (FIG. 5). The steroid hormone/steroid hormone binding protein complexes are internalised and delivered to lysosomes. There, the steroid hormone binding protein is degraded to release the steroid hormone, which enters the cytoplasm for further metabolism. In the cytoplasm, steroid hormones may be further metabolized such as testosterone to dihydrotestosterone in principal cells of the epididymis. Alternatively, the steroid hormones interact with their nuclear hormone receptor to elicit intracellular signalling.

The uptake of steroid hormones into cells presenting a steroid hormone binding protein receptor can be altered in a number of different ways according to the present invention. For example the uptake can be altered to decrease uptake of steroid hormones into the cells. This may be obtained for example by any of the following methods:

a) inhibiting the association between a steroid hormone and a steroid hormone binding protein and/or;

b) inhibiting the association between a steroid hormone binding protein and a steroid hormone binding protein receptor and/or;

c) inhibiting the association between a steroid hormone binding protein and a steroid hormone binding protein co-receptor and/or;

d) inhibiting the association between a steroid hormone binding protein receptor and a steroid hormone binding protein co-receptor.

### FIGURES

FIG. 1. Surface-plasmon resonance analysis of binding of testosterone/SHBG complexes to purified megalin.

FIG. 2. Receptor-mediated uptake of dihydrotestosterone/SHBG complexes in BN 16 cells and keratinocytes.

FIG. 3. Histological analysis of vagina tissues from wild type and megalin knock-out mice.

FIG. 4. Model of receptor-mediated endocytosis of steroid hormone/steroid hormone binding protein (carrier) complexes.

FIG. 5. Model of uptake of steroid hormone/steroid hormone binding proteins into cells via receptor and/or co-receptor mediated endocytosis.

FIG. 6. Model of the structure of examples of steroid hormone binding protein receptors.

FIG. 7. The urogenital tract of adult wild type or megalin-deficient male mouse (megalin^{-/-}). The position of the testis is indicated by dotted line. The left testis of the receptor-deficient mice is indicated by arrow.

FIG. 8. 125I-rSHBG (400 pg) was incubated with 600 μg of membrane preparations from bovine epididymis (A) or endometrium (B) in the presence of the indicated concentrations of unlabelled rSHBG. Values are given as a percentage of 125I-rSHBG bound in the absence of competitor (100%). The data represent the means of four individual experiments (±S.E.M.). Where no error bar is shown, the values are smaller than the actual symbol.

FIG. 9. Immunohistological detection of megalin in dog and rat prostate. The arrows denote localization of the receptor on the surface of epithelial cells.

### DETAILED DESCRIPTION OF THE INVENTION

Amino Acids and Nucleic Acids

Throughout the description and claims the three letter code for natural amino acids are used. Where the L or D form has not been specified it is to be understood that the amino acid in question has the natural L form, cf. Pure & Appi. Chem. Vol. (56(5) pp 595-624 (1984) or the D form, so that the peptides formed may be constituted of amino acids of L form, D form, or a sequence of mixed L forms and D forms.

Where nothing is specified it is to be understood that the C-terminal amino acid of a polypeptide of the invention exists as the free carboxylic acid, this may also be specified as “—OH”. The N-terminal amino acid of a polypeptide comprise a free amino-group, this may also be specified as “H—”.

Where nothing else is specified amino acid can be selected from any amino acid, whether naturally occurring or not, such as alfa amino acids, beta amino acids, and/or gamma amino acids. Accordingly, the group comprises but are not limited to: Ala, Val, Leu, Ile, Pro, Phe, Trp, Met, Gly, Ser, Thr, Cys, Tyr, Asn, Gln, Asp, Glu, Lys, Arg, His, Aib, Nal, Sar, Orn, Lysine analogues DAP and DAPA.

The term “nucleic acid” is meant to encompass DNA and RNA as well as derivatives thereof such as peptide nucleic acids (PNA) or locked nucleic acids (LNA) throughout the description.

Steroid Hormones

The steroid hormone according to the present invention can be any steroid hormone. The term steroid hormone covers a group of small hydrophobic molecules, which are structurally derived from cholesterol. For example such a steroid hormone can be selected from the group consisting of androgens, estrogens, progestogens and corticoids.

Androgens can for example be selected from the group consisting of testosterone, dihydrotestosterone, androstenediol, androstenedione, dehydroepiandrosterone (DHEA), dehydroepiandrosterone sulfate (DHEA-S) and derivatives thereof.

Estrogens can for example be selected from the group consisting of estrion, estradiol, estriol and derivatives thereof.

Progestogens can for example be selected from the group comprising progesterone, 17-hydroxy-progesterone, pregnenolone, 17-hydroxy-pregnenolone and derivatives thereof.

Corticoids can for example be selected from the group consisting of glucocorticoid, mineralcorticoid, cortisol, 11-deoxy-cortisol, corticosterone, 1-deoxy-corticosterone, 18-hydroxy-corticosterone, aldosterone and derivatives thereof.

Preferably, the steroid hormone is selected from the group consisting of androgens and estrogens. More preferably, the steroid hormone is selected from testosterone, dihydrotestosterone, estrion, estradiol and derivatives thereof.

The steroid hormone according to the present invention may be naturally occurring steroid hormones, it may be recombinantly produced steroid hormones or derivatives thereof or it may be synthetic derivatives of steroid hormones.

Steroid Hormone Binding Protein

The steroid hormone binding protein according to the present invention can be any steroid hormone binding protein.

For example the steroid hormone binding protein is selected from the group consisting of sex hormone binding globulin, corticosteroid-binding globulin, Clara cell secretory protein (CCSP), apolipoprotein D (apoD) and 24p3. Preferably, the steroid hormone binding protein is the sex hormone binding globulin. Furthermore, it is preferred that the steroid hormone binding protein according to the present invention is a human steroid hormone binding protein.

Preferred steroid hormone binding proteins according to the present invention are listed in table 1.

TABLE 1

| | accession # |
|---|---|
| Steroid hormone binding protein | (human) |
| sex hormone binding globulin | cDNA: |
| (SHBG) | XM_008233 |
| | NM_001040 |
| | Gene: |
| | M31651 |
| corticosteroid binding globulin | cDNA: |
| (CBG) | XM_007483 |
| | J02943 |
| | Gene: |
| | M31662 |
| apolipoprotein D | cDNA: |
| (apoD) | XM_003067 |
| | NM_001647 |
| 24p3 | cDNA: |
| or | XM_005667 |
| lipocalin 2 | NM_005564 |
| | Gene: |
| | NT_008581 |
| Clara cell secretory protein | cDNA: |
| (CCSP) | XM_006407 |
| or | Gene: |
| Uteroglobin | NT_009314 |

Steroid Hormone Binding Protein Receptor/Co-Receptor

The term “steroid hormone binding protein receptor” is meant to encompass any receptor, which has a steroid hormone binding protein as ligand. Preferably, the steroid hormone binding protein receptor is selected from the group consisting of receptors belonging to the low density lipoprotein (LDL) receptor gene family.

The present inventors have shown that the receptor according to the invention relates to the low density lipoprotein (LDL) receptor gene family comprising receptors selected from the group consisting of VLDL receptor, LRP-1-B, MEGF-7, ApoE receptor-2, chicken VTG receptor, Drosophila Y1 protein, LDL receptor-related protein (LRP), gp 330/megalin and C. elegans gp330/megalin-related protein. However, in respect of the present invention it has been shown that not all members of the family may be included, since for example the LDL-receptor does not exhibit sufficient steroid hormone binding protein receptor properties.

Accordingly, the steroid hormone binding protein receptor according to the present invention preferably has a binding affinity for a steroid hormone binding protein, which is at least 4 fold above the binding affinity of the LDL-receptor to said steroid hormone binding protein, such as at least 5 fold, for example at least 6 fold, such as at least 7.5 fold, for example at least 10 fold, preferably at least between 5 and 10 fold above the binding affinity of the LDL-receptor to said steroid hormone binding protein.

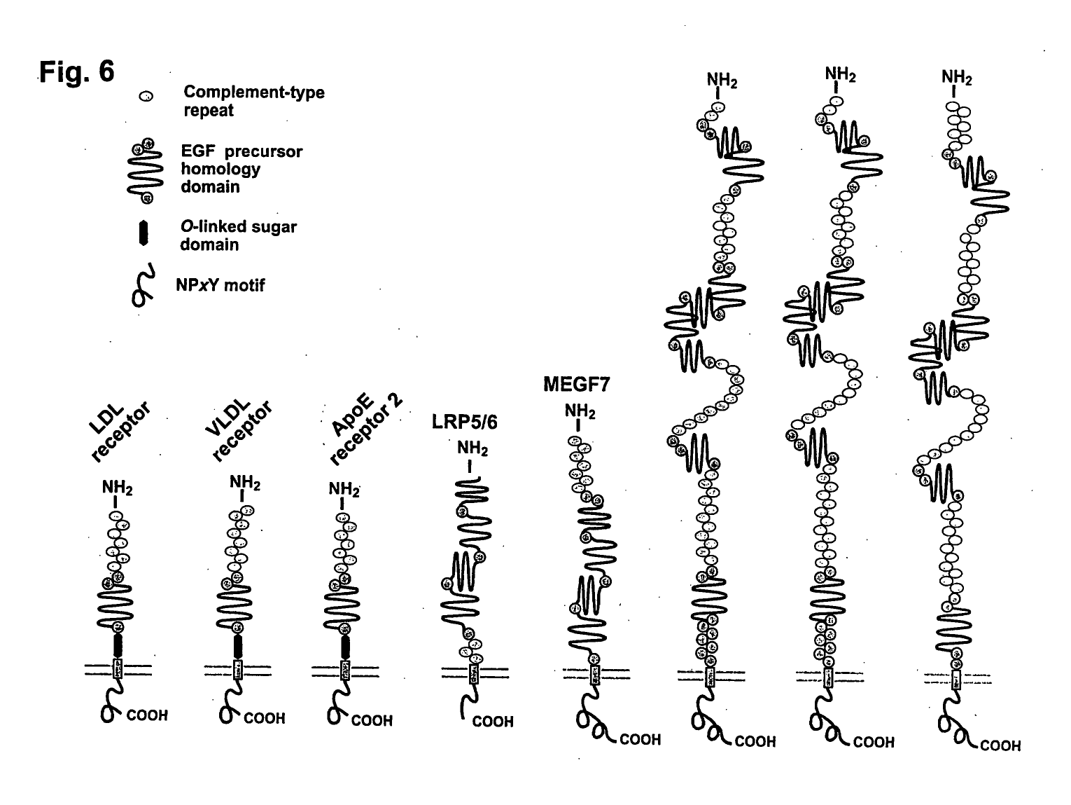

Preferred steroid hormone binding protein receptors according to the present invention are listed in table 2. Structural models of examples of steroid hormone binding protein receptors (LDL receptor, LRP5/6, LRP, megalin, VLDL receptor, MGEF-7, LRP1 B and ApoE receptor-2) are given in FIG. 6.

Preferably, the steroid hormone binding protein receptor is selected from the group consisting of LRP, megalin, VLDL receptor, MGEF-7, LRP1 B and ApoE receptor-2.

More preferably, the steroid hormone binding protein receptor is selected from the group consisting of ApoE receptor-2 and megalin. Yet more preferably, the steroid hormone binding protein receptor is megalin.

TABLE 2

| | accession # |
|---|---|
| receptor name(s) | (human) |
| LRP1 | cDNA: |
| LDL receptor-related protein | X13916 NM_002332 |
| alpha-2-macroglobulin receptor | gene: |
| | AH003324 |
| LRP2 | cDNA: |
| Megalin | U33837 |
| gp330/gp600 | gene: |
| | NT_002176 |
| apolipoprotein E receptor 2 | cDNA: |
| ApoE receptor 2 | D50678 |
| LRP8 | gene: |
| | SEG_D86389S |
| very low density lipoprotein | cDNA: |
| receptor | D16493 |
| VLDL receptor | gene: |
| | SEG_HUMVLDLR |
| LRP1B | cDNA: |
| | NM_018557 |
| MGEF-7 | cDNA: |
| | AB011540 |

Furthermore, it is preferred that the steroid hormone binding protein receptor according to the present invention is a human steroid hormone binding protein receptor.

In certain cases uptake of steroid hormone binding protein into cells requires in addition to a steroid hormone binding protein receptor also a steroid hormone binding protein co-receptor. In particular, association between a steroid hormone binding protein and a steroid hormone binding protein co-receptor may be required for cellular uptake. Steroid hormone binding protein co-receptors within the scope of the present invention may for example be cubilin (see table 3).

Cubilin was first identified as a receptor for IF-B₁₂ complex in the terminal ileum (Birn et al., 1997). Cubilin directly associates with megalin (Moestrup et al., 1998), and this molecular cooperation likely provides the basis for internalization of ligands bound to cubilin. Thus, cubilin with bound ligand may undergo megalin-mediated endocytosis, unload its cargo in lysosomes, and recycle back to the plasma membrane together with megalin. For example it has been demonstrated that cubilin greatly facilitates the endocytic uptake of 25(OH)D3 DBP by sequestering the complex on the cell surface before internalization via megalin (Nykjaer et al, 2001).

TABLE 3

| | accession # |
|---|---|
| Protein name(s) | (human) |
| Cubilin | cDNA: |
| | XM_011904 |
| | gene: |
| | NT_008682 (Homo |
| | sapiens chromosome |
| | 10 working draft |
| | sequence segment) |

TABLE 4

| | |
|---|---|
| RAP | cDNA: |
| Low density lipoprotein-related | XM_003315 |
| protein-associated protein 1 | Gene: |
| Alpha-2-macroglobulin receptor- | AH006949 |
| associated protein | |
| LRPAP1 | |
| Lipoprotein receptor associated | |
| protein | |

Cells

The cells presenting the steroid hormone binding protein receptors according to the present invention can be cells which have been grown in a tissue culture in vitro or they may be cells comprised within an individual. Said individual is preferably a mammal, more preferably a human being. Depending on the steroid hormone of the specific application of the present invention, the cells should be selected so that they preferably are responsive to said steroid hormone.

In particular, the cells may be selected from the group consisting of prostate cells, cells in epididymis, endometrial cells, ovarial cells, breast parenchymal cells, prostate carcinoma cells and breast carcinoma cells.

Any cells that have been genetically modified to express the steroid hormone binding protein receptor or co-receptor as well as the nuclear receptor may be used. Such transfected cells are preferably eukaryotic cells, more preferably mammalian cells, most preferably human cells.

Assay

The present invention does not depend on any particular type of assay for measuring binding and/or uptake into cells of steroid hormones by steroid hormone binding protein receptors. Any assay capable of measuring binding and/or uptake into cells of the steroid hormones by steroid hormone binding protein receptors can be used in conjunction with the present invention. Assays based on a specific recognition between a steroid hormone and a steroid hormone binding protein and/or a steroid hormone binding protein and a steroid hormone binding protein receptor or a steroid hormone binding protein co-receptor according to the specific application of the present invention are preferred.

For example an assay could be qualitative and/or quantitative assays involving the use of immunoreactive species, i.e. antigens, haptens and antibodies or fragments thereof.

The assay according to the present invention may in one embodiment employ standard immunohistochemical or cytochemical detection procedures, or suitable modifications thereof, for the detection of steroid hormone. Accordingly, the invention may employ any assay resulting in the recognition of steroid hormone by an immunochemical reaction with a specific so-called primary antibody capable of reacting exclusively with the steroid hormone.

The primary antibody may be labelled with an appropriate label capable of generating—directly or indirectly—a detectable signal. The label is preferably an enzyme, a radioactive isotope, a fluorescent group, a dye, a chemiluminescent molecule or a heavy metal such as gold.

In another embodiment, the invention employs the detection of the primary antibody by immunochemical reaction with specific so-called secondary antibodies capable of reacting specifically with the primary antibodies. In this case the secondary antibodies can be labelled with an appropriate label such as an enzyme, a radioactive isotope, a fluorescent group, a dye, a chemiluminescent molecule or a heavy metal such as gold.

In yet another embodiment, the present invention employs a so-called linker antibody as a means of detection of the steroid hormone. This embodiment exploits that the immunochemical reaction between the steroid hormone and the primary antibody is mediated by another immunochemical reaction involving the specific linker antibody capable of reacting simultaneously with both the primary antibody as well as another antibody to which enzymes have been attached via an immunochemical reaction, or via covalent coupling and the like.

In yet another embodiment according to the present invention, the immunochemical reaction between the steroid hormone and the primary antibody, or alternatively, between the primary antibody and the secondary antibody, is detected by means of a binding of pairs of complementary molecules other than antigens and antibodies. A complementary pair such as e.g. biotin and streptavidin is preferred. In this embodiment, one member of the complementary pair is attached to the primary or secondary antibody, and the other member of the complementary pair is contacted by any suitable label such as e.g. an enzymes, a radioactive isotope, a fluorescent group, a dye or a heavy metal such as gold.

In a preferred embodiment the assay takes advantage of the specific interaction between steroid hormones and steroid hormone binding proteins and/or the specific interaction between steroid hormone binding proteins and steroid hormone binding protein receptors or a steroid hormone binding protein co-receptors. Such an assay involves the use of one or more purified species selected from the group consisting of steroid hormones, steroid hormone binding proteins, steroid hormone binding protein receptors and fragments thereof and functional homologues thereof.

In such an assay at least one of either steroid hormones, steroid hormone binding proteins, steroid hormone binding protein receptors, a steroid hormone binding protein co-receptors or fragments thereof is labelled with an appropriate label capable of generating—directly or indirectly—a detectable signal. The label is preferably an enzyme, a radioactive isotope, a fluorescent group, a dye, a chemiluminescent molecule or a heavy metal such as gold. However, it is also contained within the present invention that more than one such as for example two or for example three, such as all four of steroid hormones, steroid hormone binding proteins, steroid hormone binding protein receptors, a steroid hormone binding protein co-receptor or fragments thereof are labelled with a detectable label: Said detectable label may be the same for two or more species or it may be a different label for each species.

It is preferred that at least one of either steroid hormones, steroid hormone binding proteins, steroid hormone binding protein receptors, a steroid hormone binding protein co-receptors or fragments thereof is immobilised on a solid support. More preferably, steroid hormone binding protein receptors or fragments thereof are immobilised. Immobilisation may be achieved by any standard method known to the person skilled in the art useful for the solid support of the specific application. The solid support can be any solid support suitable for such an assay. In some embodiments of the present invention a suitable solid support is for example a microtiter plate or a carboxymethylated dextran-gold surface.

Preferably, such an assay is a non-radioactive microtiter plate assay using purified steroid hormone binding protein receptors or fragments thereof immobilised on the plate surface.

In one preferred embodiment of the present invention the assay comprises the steps of

providing a non-radioactive microtiter plate having purified steroid hormone binding protein receptors or fragments thereof immobilised on the plate surface,

incubating the plate with the compound to be tested, and

incubating the plate with fluorescent labelled steroid binding proteins or fragments thereof and/or with fluorescent labelled steroid hormones,

determining the amount of fluorescence of the plate.

However, in another preferred embodiment the solid support is a carboxymethylated dextran-gold surface and the assay is a surface plasmon resonance analysis comprising the steps of

i) Immobilising purified steroid hormone binding protein receptor and/or fragments thereof on a carboxymethylated dextran-gold surface.

ii) Injecting the compound to be tested over the dextran-gold surface

iii) measuring the kinetics of binding in real time.

In one embodiment of the present invention the assay is a biological assay. For example such an assay comprises the steps of

a) Providing a cell which comprises a steroid hormone protein binding receptor, a steroid hormone nuclear receptor and a first nucleic acid sequence encoding a directly or indirectly detectable protein, said first nucleic acid sequence being operably linked to a second nucleic acid sequence, which can direct transcription depending on the presence of steroid hormone nuclear receptor complexed with steroid hormone.

b) Detecting said detectable protein.

c) Correlating the amount of detectable protein to the uptake of steroid hormone into said cell.

Such a cell could be any cell suitable for such an assay, for example the cell could be a cell of mammalian origin cultured in vitro or it could be a cell of mammalian origin comprised within a mammal. However, the cell could also be another cell, such as for example any other eukaryotic cell or prokaryotic cell. Examples of useful eukaryotic cells other than mammalian cells are yeast cells or an insect cells.

The steroid hormone nuclear receptor should be selected according to the individual need in a way such as it associates with the steroid hormone of the particular embodiment of the present invention.

The second nucleic acid sequence should be selected such as it can direct transcription in the cell of the particular embodiment depending on the presence of steroid hormone nuclear receptor complexed with steroid hormone. Preferably such a nucleic acid sequence comprises a minimal promoter sequence and steroid hormone nuclear receptor responsive elements. These steroid hormone nuclear receptor responsive elements should be selected according to the steroid hormone nuclear receptor of the particular embodiment.

Said detectable protein may be selected from any useful detectable protein depending on the particular assay. For example such a protein could be an enzyme or it could be a fluorescent protein. Examples of useful enzymes are luciferase or peroxidase. Examples of fluorescent proteins are green fluorescent protein (GFP), yellow fluorescent protein (YFP), blue fluorescent protein (BFP) and derivatives thereof. Preferably, the detectable protein is selected from the group consisting of luciferase and GFP.

The compound according to the present invention may be a nucleic acid sequence which potentially alters the expression of a steroid hormone binding protein receptor and/or steroid hormone binding protein co-receptor. If the compound according to the present invention is a nucleic acid sequence, which potentially is capable of decreasing the expression of a steroid hormone binding protein receptor and/or a steroid hormone binding protein co-receptor (see herein below) the assay preferably includes a cell normally expressing the steroid hormone binding protein receptor and/or the steroid hormone binding protein co-receptor.

In one embodiment such an assay comprise the following steps:

a) introducing the nucleic acid sequence into the cell expressing the steroid hormone binding protein receptor

b) detecting the presence of the steroid hormone binding protein receptor in the cell

In another embodiment the nucleic acid sequence potentially increase the expression of either steroid hormone binding protein receptor and/or steroid hormone binding protein co-receptor. In that case the assay may comprise the steps of:

a) introducing one or more nucleic acid sequences into a cell, wherein the nucleic acids encode for one or more

i) steroid hormone binding protein receptors; or

ii) steroid hormone binding protein co-receptors; or

iii) steroid hormone binding protein receptors and steroid hormone binding protein co-receptors

b) detecting the presence of steroid hormone binding protein receptor and/or steroid hormone binding protein co-receptor in said cell.

Preferably, when nucleic acid sequences according to step ii) are employed, the cell should express a steroid hormone binding protein receptor endogenously.

Nucleic acid sequences may be introduced into the cell by any standard method known to the person skilled in the art. For example transfer of a nucleic acid sequence into a cell can be accomplished by electroporation, microinjection, lipofection with for example cationic liposomes, calcium phosphate precipitation, viral transfer, retroviral transfer, adsorption or bio-ballistic transfer by for example coated gold particles. In particular, electroporation, lipofection or viral transfer are preferred methods.

Detection of the steroid hormone binding protein receptor and/or steroid hormone binding protein co-receptor in the cell can be done by a number of methods known to the person skilled in the art. For example such a method could involve immunoreactive species such as for example antibodies or fragments thereof. In particular such a method could comprise a first antibody interacting specifically with the steroid hormone binding protein receptor and/or steroid hormone binding protein co-receptor. Said first antibody could comprise a directly or indirectly detectable label or the assay could further involve a second antibody, which comprises a directly or indirectly detectable label and which reacts specifically with the first antibody. In a preferred embodiment the steroid hormone binding protein receptor and/or steroid hormone binding protein co-receptor is detected by a method which involves the use of steroid hormones and/or steroid hormone binding proteins, wherein one and/or both comprise a directly or indirectly detectable label.

The present invention also relates to methods for determining the effect of a compound on uptake of steroid hormones into cells presenting a steroid hormone binding protein receptor following administration of said compound to a mammal naturally expressing the receptor.

Such a method involves for example measuring expression of receptor mRNA or protein in tissues of the mammal or measuring receptor-mediated binding or transport of steroid hormones bound to or complexed with steroid hormone binding proteins.

Measuring expression of protein in tissues of the mammal can be done by any of the assays as outlined herein above. In particular, the assay may involve the use of immunoreactive species or the assay may involve the use of one or more purified species selected from the group consisting of steroid hormones, steroid hormone binding proteins, steroid hormone binding protein receptors, steroid hormone binding protein co-receptor and fragments thereof and functional homologues thereof.

Measuring of mRNA may be done according to any standard protocol known to the person skilled in the art. For example such an assay could be a Northern blotting assay using a nucleic acid sequence derived from the receptor of the particular embodiment as probe. The probe should be labelled with a directly or indirectly detectable label, for example a radioactive species or an enzyme. Another example of such an assay is reverse transcription polymerase chain reaction (RT-PCR). Such an RT-PCR would involve the use of specific primers chosen according to the receptor of the particular embodiment.

Determining transport of steroid hormones bound to or complexed with steroid hormone binding proteins can be done by a number of assays including the assays outlined herein above. In particular, the assay may involve the use of immunoreactive species or the assay may involve the use of one or more purified species selected from the group consisting of steroid hormones, steroid hormone binding proteins, steroid hormone binding protein receptors, steroid hormone binding protein co-receptors and fragments thereof and functional homologues thereof. Furthermore, such an assay may be a biological assay as outlined herein above.

Animals Lacking Expression of a Steroid Hormone Binding Protein Receptor

In one aspect the present invention relates to animals lacking expression of one or more steroid hormone binding protein receptor(s) and/or co-receptor(s) and/or steroid hormone binding proteins and to the use of such animals for example for testing the compounds of the invention.

For example, said animals may have been genetically engineered so they lack or carry a non-functional gene for said steroid hormone binding protein receptor and/or co-receptor or for the steroid hormone binding protein itself. An example of an animal lacking a steroid hormone binding protein receptor is described in example 3.

In another example, said animals may have been genetically engineered so they lack or carry a non-functional gene for said steroid hormone binding protein receptor and/or co-receptor only in specific selected tissues, i.e. tissue specific knock out. An example of tissue specific knock out is given in example 6.

The animal may be any animal such as a mammal, however frequently it is preferred that the animal is a rodent, preferably a mouse.

In one embodiment of the present invention said animal may be used in a method for determining the effect of a compound on uptake of steroid hormones into cells via a steroid hormone binding protein receptor. In particular said animal may be used as a negative control in such a method.

For example, the method may comprise the step of administering said compound to a mammal, such as a mouse lacking expression of said steroid hormone binding protein receptor. For example, said mammal may only lack expression of said steroid hormone binding protein receptor in one or more selected tissues. The method may further comprise a step of measuring the uptake of one or more steroid hormones or one or more steroid hormone/steroid hormone binding protein complexes into cells of said animal. The method may further comprise a step of comparing said uptake with the uptake of one or more steroid hormones or one or more steroid hormone/steroid hormone binding protein complexes into cells of an animal expressing said steroid hormone binding protein receptor.

Compounds

The compounds according to the present invention, which alter the uptake of steroid hormone into cells presenting a steroid hormone binding protein receptor are preferably identified by the methods outlined herein above. The compounds could for example be polypeptides, nucleic acids or small organic molecules.

In one embodiment the compound competitively inhibits the binding or complexing of a steroid hormone to a steroid hormone binding protein. Such a compound could for example be a compound that specifically interacts with either the steroid hormone or with the steroid hormone binding protein in a way that sterically inhibits further association with either the steroid hormone or with the steroid hormone binding protein.

In another embodiment the compound competitively inhibits the binding of a steroid hormone binding protein to a steroid hormone binding protein receptor. Such a compound could for example be a compound that specifically interacts with either the steroid hormone protein or with the steroid hormone binding protein receptor in a way that sterically inhibits further association with either the steroid hormone protein or with the steroid hormone binding protein receptor.

In yet another embodiment the compound competitively inhibits the binding of a steroid hormone binding protein to a steroid hormone binding protein co-receptor.

In a still further embodiment of the present invention the compound increases the uptake of the steroid hormone.

In yet a further embodiment the compound alters dimerisation of steroid hormone binding proteins, such as it increases dimerisation of steroid hormone binding proteins.

In still another embodiment the compound has the potential to alter the expression of a steroid hormone binding protein receptor in a cell. For example, the compound may decrease the expression of a steroid hormone binding protein receptor in a cell normally expressing such a steroid hormone binding protein receptor or alternatively the compound may increase the expression of a steroid hormone binding protein receptor in a cell.

Furthermore, the compound according to the present invention may have the potential to alter the expression of a steroid hormone binding protein co-receptor in a cell. For example, the compound may decrease the expression of a steroid hormone binding protein co-receptor in a cell normally expressing such a steroid hormone binding protein receptor or alternatively the compound may increase the expression of a steroid hormone binding protein co-receptor in a cell.

The compound according to the present invention can be selected from a library of naturally occurring and synthetic compounds, which are randomly tested for alteration of the binding.

In one embodiment of the present invention, the compound is a polypeptide. For example such polypeptides could be selected from the group consisting of steroid hormone binding protein receptor domains and fragments thereof, steroid hormone binding protein co-receptor domains and fragments thereof, natural steroid hormone binding protein receptor ligands, modified steroid hormone binding proteins or fragments thereof, fragments of steroid hormone binding proteins, steroid hormone binding protein receptor antagonists, such as receptor associated protein (RAP; see table 4), and functional homologues of any of these.

In one preferred embodiment of the present invention the compound is a steroid hormone binding protein receptor domain. Preferably, said steroid hormone binding protein receptor domain is capable of binding a steroid hormone binding protein.

It has been shown that complement type repeats of LRP are capable of binding a protein designated RAP. More specifically everyone of the 8 complement type repeats of cluster 11 of LRP are able to bind RAP with the exception of repeat 8, which differs from the rest in that it lacks a negatively charged amino acid (Andersen et al., 2000, J. Biol. Chem. 275:21017-21024).

Accordingly, it is preferred that the steroid hormone binding protein receptor domain comprise at least one complement type repeat, more preferably, at least two complement type repeats. However, it is also contained within the present invention that the steroid hormone binding protein receptor domain comprise 2 complement type repeats, such as 3 complement type repeats, for example 4 complement type repeats, such as 5 complement type repeats, for example 6 complement type repeats, such as 7 complement type repeats, for example 8 complement type repeats, such as 9 complement type repeats, for example 10 complement type repeats, such as 11 complement type repeats, for example more than 11 complement type repeats. In a preferred embodiment the steroid hormone binding protein receptor domain comprises 2 complement-type repeats.

A model of the domain structure of a number of steroid hormone binding protein receptors is given in FIG. 6.

The complement type repeats preferably comprise approximately 40 amino acids, more preferably 40 amino acids, which comprise the following pattern of three disulfide bridges: CysI-CysII, Cysil-CysV and CysIV-Cys VI, wherein the roman numbers designate relative sequence position of conserved cysteine residues within the complement repeat. Furthermore, each complement type repeat preferably comprises a negatively charged amino acid residue, preferably, said negatively charged amino acid residue is placed between CysIV and CysV, more preferably it is placed in the centre position between CysIV and CysV. The negatively charged acidic amino acid residue is preferably selected from the group consisting of Asp and Glu.

In one embodiment the steroid hormone binding protein receptor domain comprises fragments of the MEGF7 polypeptide sequence (SEQ ID NO: 2). For example such a fragment may comprise amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 0.600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 975, for example amino acid 950 to 1000, such as amino acid 975 to 1025, 1000 to 1050, such as amino acid 1025 to 1075, for example amino acid 1050 to 1100, such as amino acid 1075 to 1125, for example amino acid 1100 to 1150, such as amino acid 1125 to 1175, for example amino acid 1150 to 1200, such as amino acid 1175 to 1225, for example amino acid 1200 to 1250, such as amino acid 1225 to 1275, for example amino acid 1250 to 1300, such as amino acid 1275 to 1325, for example amino acid 1300 to 1350, such as amino acid 1325 to 1375, for example amino acid 1350 to 1400, such as amino acid 1375 to 1425, for example amino acid 1400 to 1450, such as amino acid 1425 to 1475, for example amino acid 1450 to 1500, such as amino acid 1475 to 1525, for example amino acid 1500 to 1550, such as amino acid 1525 to 1576 of the MEGF7 polypeptide sequence (SEQ ID NO: 2).

In another embodiment the steroid hormone binding protein receptor domain essentially consists of or preferably consists of fragments of the MEGF7 polypeptide sequence (SEQ ID NO: 2). For example such a fragment may consist of amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 975, for example amino acid 950 to 1000, such as amino acid 975 to 1025, 1000 to 1050, such as amino acid 1025 to 1075, for example amino acid 1050 to 1100, such as amino acid 1075 to 1125, for example amino acid 1100 to 1150, such as amino acid 1125 to 1175, for example amino acid 1150 to 1200, such as amino acid 1175 to 1225, for example amino acid 1200 to 1250, such as amino acid 1225 to 1275, for example amino acid 1250 to 1300, such as amino acid 1275 to 1325, for example amino acid 1300 to 1350, such as amino acid 1325 to 1375, for example amino acid 1350 to 1400, such as amino acid 1375 to 1425, for example amino acid 1400 to 1450, such as amino acid 1425 to 1475, for example amino acid 1450 to 1500, such as amino acid 1475 to 1525, for example amino acid 1500 to 1550, such as amino acid 1525 to 1576 of the MEGF7 polypeptide sequence (SEQ ID NO: 2).

In another embodiment the steroid hormone binding protein receptor domain comprise fragments of the megalin polypeptide sequence (SEQ ID NO: 3). For example such a fragment may comprise amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 975, for example amino acid 950 to 1000, such as amino acid 975 to 1025, 1000 to 1050, such as amino acid 1025 to 1075, for example amino acid 1050 to 1100, such as amino acid 1075 to 1125, for example amino acid 1100 to 1150, such as amino acid 1125 to 1175, for example amino acid 1150 to 1200, such as amino acid 1175 to 1225, for example amino acid 1200 to 1250, such as amino acid 1225 to 1275, for example amino acid 1250 to 1300, such as amino acid 1275 to 1325, for example amino acid 1300 to 1350, such as amino acid 1325 to 1375, for example amino acid 1350 to 1400, such as amino acid 1375 to 1425, for example amino acid 1400 to 1450, such as amino acid 1425 to 1475, for example amino acid 1450 to 1500, such as amino acid 1475 to 1525, for example amino acid 1500 to 1550, such as amino acid 1525 to 1575, for example amino acid 1550 to 1600, such as amino acid 1575 to 1625, for example amino acid 1600 to 1650, such as amino acid 1625 to 1675, for example amino acid 1650 to. 1700, such as amino acid 1675 to 1725, for example amino acid 1700 to 1750, such as amino acid 1725 to 1775, for example amino acid 1750 to 1800, such as amino acid 1775 to 1825, for example amino acid 1800 to 1850, such as amino acid 1825 to 1875, for example amino acid 1850 to 1900, such as amino acid 1875 to 1925, for example amino acid 1900 to 1950, such as amino acid 1925 to 1975, for example amino acid 1950 to 2000, such as amino acid 1975 to 2025, amino acid 2001 to 2050, such as amino acid 2025 to 2075, for example amino acid 2050 to 2100, such as amino acid 2075 to 2125, for example amino acid 2100 to 2150, such as amino acid 2125 to 2175, for example amino acid 2150 to 2200, such as amino acid 2175 to 2225, for example amino acid 2200 to 2250, such as amino acid 2225 to 2275, for example amino acid 2250 to 2300, such as amino acid 2275 to 2325, for example amino acid 2300 to 2350, such as amino acid 2325 to 2375, for example amino acid 2350 to 2400, such as amino acid 2375 to 2425, for example amino acid 2400 to 2450, such as amino acid 2425 to 2475, for example amino acid 2450 to 2500, such as amino acid 2475 to 2525, for example amino acid 2500 to 2550, such as amino acid 2525 to 2575, for example amino acid 2550 to 2600, such as amino acid 2575 to 2625, for example amino acid 2600 to 2650, such as amino acid 2625 to 2675, for example amino acid 2650 to 2700, such as amino acid 2675 to 2725, for example amino acid 2700 to 2750, such as amino acid 2725 to 2775, for example amino acid 2750 to 2800, such as amino acid 2775 to 2825, for example amino acid 2800 to 2850, such as amino acid 2825 to 2875, for example amino acid 2850 to 2900, such as amino acid 2875 to 2925, for example amino acid 2900 to 2950, such as amino acid 2925 to 2975, for example amino acid 2950 to 3000, such as amino acid 2975 to 3025, amino acid 3001 to 3050, such as amino acid 3025 to 3075, for example amino acid 3050 to 3100, such as amino acid 3075 to 3125, for example amino acid 3100 to 3150, such as amino acid 3125 to 3175, for example amino acid 3150 to 3200, such as amino acid 3175 to 3225, for example amino acid 3200 to 3250, such as amino acid 3225 to 3275, for example amino acid 3250 to 3300, such as amino acid 3275 to 3325, for example amino acid 3300 to 3350, such as amino acid 3325 to 3375, for example amino acid 3350 to 3400, such as amino acid 3375 to 3425, for example amino acid 3400 to 3450, such as amino acid 3425 to 3475, for example amino acid 3450 to 3500, such as amino acid 3475 to 3525, for example amino acid 3500 to 3550, such as amino acid 3525 to 3575, for example amino acid 3550 to 3600, such as amino acid 3575 to 3625, for example amino acid 3600 to 3650, such as amino acid 3625 to 3675, for example amino acid 3650 to 3700, such as amino acid 3675 to 3725, for example amino acid 3700 to 3750, such as amino acid 3725 to 3775, for example amino acid 3750 to 3800, such as amino acid 3775 to 3825, for example amino acid 3800 to 3850, such as amino acid 3825 to 3875, for example amino acid 3850 to 3900, such as amino acid 3875 to 3925, for example amino acid 3900 to 3950, such as amino acid 3925 to 3975, for example amino acid 3950 to 4000, such as amino acid 3975 to 4025, amino acid 4001 to 4050, such as amino acid 4025 to 4075, for example amino acid 4050 to 4100, such as amino acid 4075 to 4125, for example amino acid 4100 to 4150, such as amino acid 4125 to 4175, for example amino acid 4150 to 4200, such as amino acid 4175 to 4225, for example amino acid 4200 to 4250, such as amino acid 4225 to 4275, for example amino acid 4250 to 4300, such as amino acid 4275 to 4325, for example amino acid 4300 to 4350, such as amino acid 4325 to 4375, for example amino acid 4350 to 4400, such as amino acid 4375 to 4425, for example amino acid 4400 to 4450, such as amino acid 4425 to 4475, for example amino acid 4450 to 4500, such as amino acid 4475 to 4525, for example amino acid 4500 to 4550, such as amino acid 4525 to 4575, for example amino acid 4550 to 4600, such as amino acid 4575 to 4625, for example amino acid 4600 to 4655 of the megalin polypeptide sequence (SEQ ID NO: 3).

In another embodiment the steroid hormone binding protein receptor domain essentially consists of or preferably consists of fragments of the megalin polypeptide sequence (SEQ ID NO: 3). For example such a fragment may consist of amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 975, for example amino acid 950 to 1000, such as amino acid 975 to 1025, 1000 to 1050, such as amino acid 1025 to 1075, for example amino acid 1050 to 1100, such as amino acid 1075 to 1125, for example amino acid 1100 to 1150, such as amino acid 1125 to 1175, for example amino acid 1150 to 1200, such as amino acid 1175 to 1225, for example amino acid 1200 to 1250, such as amino acid 1225 to 1275, for example amino acid 1250 to 1300, such as amino acid 1275 to 1325, for example amino acid 1300 to 1350, such as amino acid 1325 to 1375, for example amino acid 1350 to 1400, such as amino acid 1375 to 1425, for example amino acid 1400 to 1450, such as amino acid 1425 to 1475, for example amino acid 1450 to 1500, such as amino acid 1475 to 1525, for example amino acid 1500 to 1550, such as amino acid 1525 to 1575, for example amino acid 1550 to 1600, such as amino acid 1575 to 1625, for example amino acid 1600 to 1650, such as amino acid 1625 to 1675, for example amino acid 1650 to 1700, such as amino acid 1675 to 1725, for example amino acid 1700 to 1750, such as amino acid 1725 to 1775, for example amino acid 1750 to 1800, such as amino acid 1775 to 1825, for example amino acid 1800 to 1850, such as amino acid 1825 to 1875, for example amino acid 1850 to 1900, such as amino acid 1875 to 1925, for example amino acid 1900 to 1950, such as amino acid 1925 to 1975, for example amino acid 1950 to 2000, such as amino acid 1975 to 2025, amino acid 2001 to 2050, such as amino acid 2025 to 2075, for example amino acid 2050 to 2100, such as amino acid 2075 to 2125, for example amino acid 2100 to 2150, such as amino acid 2125 to 2175, for example amino acid 2150 to 2200, such as amino acid 2175 to 2225, for example amino acid 2200 to 2250, such as amino acid 2225 to 2275, for example amino acid 2250 to 2300, such as amino acid 2275 to 2325, for example amino acid 2300 to 2350, such as amino acid 2325 to 2375, for example amino acid 2350 to 2400, such as amino acid 2375 to 2425, for example amino acid 2400 to 2450, such as amino acid 2425 to 2475, for example amino acid 2450 to 2500, such as amino acid 2475 to 2525, for example amino acid 2500 to 2550, such as amino acid 2525 to 2575, for example amino acid 2550 to 2600, such as amino acid 2575 to 2625, for example amino acid 2600 to 2650, such as amino acid 2625 to 2675, for example amino acid 2650 to 2700, such as amino acid 2675 to 2725, for example amino acid 2700 to 2750, such as amino acid 2725 to 2775, for example amino acid 2750 to 2800, such as amino acid 2775 to 2825, for example amino acid 2800 to 2850, such as amino acid 2825 to 2875, for example amino acid 2850 to 2900, such as amino acid 2875 to 2925, for example amino acid 2900 to 2950, such as amino acid 2925 to 2975, for example amino acid 2950 to 3000, such as amino acid 2975 to 3025, amino acid 3001 to 3050, such as amino acid 3025 to 3075, for example amino acid 3050 to 3100, such as amino acid 3075 to 3125, for example amino acid 3100 to 3150, such as amino acid 3125 to 3175, for example amino acid 3150 to 3200, such as amino acid 3175 to 3225, for example amino acid 3200 to 3250, such as amino acid 3225 to 3275, for example amino acid 3250 to 3300, such as amino acid 3275 to 3325, for example amino acid 3300 to 3350, such as amino acid 3325 to 3375, for example amino acid 3350 to 3400, such as amino acid 3375 to 3425, for example amino acid 3400 to 3450, such as amino acid 3425 to 3475, for example amino acid 3450 to 3500, such as amino acid 3475 to 3525, for example amino acid 3500 to 3550, such as amino acid 3525 to 3575, for example amino acid 3550 to 3600, such as amino acid 3575 to 3625, for example amino acid 3600 to 3650, such as amino acid 3625 to 3675, for example amino acid 3650 to 3700, such as amino acid 3675 to 3725, for example amino acid 3700 to 3750, such as amino acid 3725 to 3775, for example amino acid 3750 to 3800, such as amino acid 3775 to 3825, for example amino acid 3800 to 3850, such as amino acid 3825 to 3875, for example amino acid 3850 to 3900, such as amino acid 3875 to 3925, for example amino acid 3900 to 3950, such as amino acid 3925 to 3975, for example amino acid 3950 to 4000, such as amino acid 3975 to 4025, amino acid 4001 to 4050, such as amino acid 4025 to 4075, for example amino acid 4050 to 4100, such as amino acid 4075 to 4125, for example amino acid 4100 to 4150, such as amino acid 4125 to 4175, for example amino acid 4150 to 4200, such as amino acid 4175 to 4225, for example amino acid 4200 to 4250, such as amino acid 4225 to 4275, for example amino acid 4250 to 4300, such as amino acid 4275 to 4325, for example amino acid 4300 to 4350, such as amino acid 4325 to 4375, for example amino acid 4350 to 4400, such as amino acid 4375 to 4425, for example amino acid 4400 to 4450, such as amino acid 4425 to 4475, for example amino acid 4450 to 4500, such as amino acid 4475 to 4525, for example amino acid 4500 to 4550, such as amino acid 4525 to 4575, for example amino acid 4550 to 4600, such as amino acid 4575 to 4625, for example amino acid 4600 to 4655 of the megalin polypeptide sequence (SEQ ID NO: 3).

In another embodiment the steroid hormone binding protein receptor domain comprises fragments of the LRP1B polypeptide sequence (SEQ ID NO: 4). For example such a fragment may comprise amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 975, for example amino acid 950 to 1000, such as amino acid 975 to 1025, 1000 to 1050, such as amino acid 1025 to 1075, for example amino acid 1050 to 1100, such as amino acid 1075 to 1125, for example amino acid 1100 to 1150, such as amino acid 1125 to 1175, for example amino acid 1150 to 1200, such as amino acid 1175 to 1225, for example amino acid 1200 to 1250, such as amino acid 1225 to 1275, for example amino acid 1250 to 1300, such as amino acid 1275 to 1325, for example amino acid 1300 to 1350, such as amino acid 1325 to 1375, for example amino acid 1350 to 1400, such as amino acid 1375 to 1425, for example amino acid 1400 to 1450, such as amino acid 1425 to 1475, for example amino acid 1450 to 1500, such as amino acid 1475 to 1525, for example amino acid 1500 to 1550, such as amino acid 1525 to 1575, for example amino acid 1550 to 1600, such as amino acid 1575 to 1625, for example amino acid 1600 to 1650, such as amino acid 1625 to 1675, for example amino acid 1650 to 1700, such as amino acid 1675 to 1725, for example amino acid 1700 to 1750, such as amino acid 1725 to 1775, for example amino acid 1750 to 1800, such as amino acid 1775 to 1825, for example amino acid 1800 to 1850, such as amino acid 1825 to 1875, for example amino acid 1850 to 1900, such as amino acid 1875 to 1925, for example amino acid 1900 to 1950, such as amino acid 1925 to 1975, for example amino acid 1950 to 2000, such as amino acid 1975 to 2025, amino acid 2001 to 2050, such as amino acid 2025 to 2075, for example amino acid 2050 to 2100, such as amino acid 2075 to 2125, for example amino acid 2100 to 2150, such as amino acid 2125 to 2175, for example amino acid 2150 to 2200, such as amino acid 2175 to 2225, for example amino acid 2200 to 2250, such as amino acid 2225 to 2275, for example amino acid 2250 to 2300, such as amino acid 2275 to 2325, for example amino acid 2300 to 2350, such as amino acid 2325 to 2375, for example amino acid 2350 to 2400, such as amino acid 2375 to 2425, for example amino acid 2400 to 2450, such as amino acid 2425 to 2475, for example amino acid 2450 to 2500, such as amino acid 2475 to 2525, for example amino acid 2500 to 2550, such as amino acid 2525 to 2575, for example amino acid 2550 to 2600, such as amino acid 2575 to 2625, for example amino acid 2600 to 2650, such as amino acid 2625 to 2675, for example amino acid 650 to 700, such as amino acid 2675 to 2725, for example amino acid 2700 to 2750, such as amino acid 2725 to 2775, for example amino acid 2750 to 2800, such as amino acid 2775 to 2825, for example amino acid 2800 to 2850, such as amino acid 2825 to 2875, for example amino acid 2850 to 2900, such as amino acid 2875 to 2925, for example amino acid 2900 to 2950, such as amino acid 2925 to 2975, for example amino acid 2950 to 3000, such as amino acid 2975 to 3025, amino acid 3001 to 3050, such as amino acid 3025 to 3075, for example amino acid 3050 to 3100, such as amino acid 3075 to 3125, for example amino acid 3100 to 3150, such as amino acid 3125 to 3175, for example amino acid 3150 to 3200, such as amino acid 3175 to 3225, for example amino acid 3200 to 3250, such as amino acid 3225 to 3275, for example amino acid 3250 to 3300, such as amino acid 3275 to 3325, for example amino acid 3300 to 3350, such as amino acid 3325 to 3375, for example amino acid 3350 to 3400, such as amino acid 3375 to 3425, for example amino acid 3400 to 3450, such as amino acid 3425 to 3475, for example amino acid 3450 to 3500, such as amino acid 3475 to 3525, for example amino acid 3500 to 3550, such as amino acid 3525 to 3575, for example amino acid 3550 to 3600, such as amino acid 3575 to 3625, for example amino acid 3600 to 3650, such as amino acid 3625 to 3675, for example amino acid 3650 to 3700, such as amino acid 3675 to 3725, for example amino acid 3700 to 3750, such as amino acid 3725 to 3775, for example amino acid 3750 to 3800, such as amino acid 3775 to 3825, for example amino acid 3800 to 3850, such as amino acid 3825 to 3875, for example amino acid 3850 to 3900, such as amino acid 3875 to 3925, for example amino acid 3900 to 3950, such as amino acid 3925 to 3975, for example amino acid 3950 to 4000, such as amino acid 3975 to 4025, amino acid 4001 to 4050, such as amino acid 4025 to 4075, for example amino acid 4050 to 4100, such as amino acid 4075 to 4125, for example amino acid 4100 to 4150, such as amino acid 4125 to 4175, for example amino acid 4150 to 4200, such as amino acid 4175 to 4225, for example amino acid 4200 to 4250, such as amino acid 4225 to 4275, for example amino acid 4250 to 4300, such as amino acid 4275 to 4325, for example amino acid 4300 to 4350, such as amino acid 4325 to 4375, for example amino acid 4350 to 4400, such as amino acid 4375 to 4425, for example amino acid 4400 to 4450, such as amino acid 4425 to 4475, for example amino acid 4450 to 4500, such as amino acid 4475 to 4525, for example amino acid 4500 to 4550, such as amino acid 4525 to 4575, for example amino acid 4550 to 4599 of the LRP1 B polypeptide sequence (SEQ ID NO: 4).

In another embodiment the steroid hormone binding protein receptor domain essentially consists of or preferably consists of fragments of the LRP1 B polypeptide sequence (SEQ ID NO: 4). For example such a fragment may consist of amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 975, for example amino acid 950 to 1000, such as amino acid 975 to 1025, 1000 to 1050, such as amino acid 1025 to 1075, for example amino acid 1050 to 1100, such as amino acid 1075 to 1125, for example amino acid 1100 to 1150, such as amino acid 1125 to 1175, for example amino acid 1150 to 1200, such as amino acid 1175 to 1225, for example amino acid 1200 to 1250, such as amino acid 1225 to 1275, for example amino acid 1250 to 1300, such as amino acid 1275 to 1325, for example amino acid 1300 to 1350, such as amino acid 1325 to 1375, for example amino acid 1350 to 1400, such as amino acid 1375 to 1425, for example amino acid 1400 to 1450, such as amino acid 1425 to 1475, for example amino acid 1450 to 1500, such as amino acid 1475 to 1525, for example amino acid 1500 to 1550, such as amino acid 1525 to 1575, for example amino acid 1550 to 1600, such as amino acid 1575 to 1625, for example amino acid 1600 to 1650, such as amino acid 1625 to 1675, for example amino acid 1650 to 1700, such as amino acid 1675 to 1725, for example amino acid 1700 to 1750, such as amino acid 1725 to 1775, for example amino acid 1750 to 1800, such as amino acid 1775 to 1825, for example amino acid 1800 to 1850, such as amino acid 1825 to 1875, for example amino acid 1850 to 1900, such as amino acid 1875 to 1925, for example amino acid 1900 to 1950, such as amino acid 1925 to 1975, for example amino acid 1950 to 2000, such as amino acid 1975 to 2025, amino acid 2001 to 2050, such as amino acid 2025 to 2075, for example amino acid 2050 to 2100, such as amino acid 2075 to 2125, for example amino acid 2100 to 2150, such as amino acid 2125 to 2175, for example amino acid 2150 to 2200, such as amino acid 2175 to 2225, for example amino acid 2200 to 2250, such as amino acid 2225 to 2275, for example amino acid 2250 to 2300, such as amino acid 2275 to 2325, for example amino acid 2300 to 2350, such as amino acid 2325 to 2375, for example amino acid 2350 to 2400, such as amino acid 2375 to 2425, for example amino acid 2400 to 2450, such as amino acid 2425 to 2475, for example amino acid 2450 to 2500, such as amino acid 2475 to 2525, for example amino acid 2500 to 2550, such as amino acid 2525 to 2575, for example amino acid 2550 to 2600, such as amino acid 2575 to 2625, for example amino acid 2600 to 2650, such as amino acid 2625 to 2675, for example amino acid 2650 to 2700, such as amino acid 2675 to 2725, for example amino acid 2700 to 2750, such as amino acid 2725 to 2775, for example amino acid 2750 to 2800, such as amino acid 2775 to 2825, for example amino acid 2800 to 2850, such as amino acid 2825 to 2875, for example amino acid 2850 to 2900, such as amino acid 2875 to 2925, for example amino acid 2900 to 2950, such as amino acid 2925 to 2975, for example amino acid 2950 to 3000, such as amino acid 2975 to 3025, amino acid 3001 to 3050, such as amino acid 3025 to 3075, for example amino acid 3050 to 3100, such as amino acid 3075 to 3125, for example amino acid 3100 to 3150, such as amino acid 3125 to 3175, for example amino acid 3150 to 3200, such as amino acid 3175 to 3225, for example amino acid 3200 to 3250, such as amino acid 3225 to 3275, for example amino acid 3250 to 3300, such as amino acid 3275 to 3325, for example amino acid 3300 to 3350, such as amino acid 3325 to 3375, for example amino acid 3350 to 3400, such as amino acid 3375 to 3425, for example amino acid 3400 to 3450, such as amino acid 3425 to 3475, for example amino acid 3450 to 3500, such as amino acid 3475 to 3525, for example amino acid 3500 to 3550, such as amino acid 3525 to 3575, for example amino acid 3550 to 3600, such as amino acid 3575 to 3625, for example amino acid 3600 to 3650, such as amino acid 3625 to 3675, for example amino acid 3650 to 3700, such as amino acid 3675 to 3725, for example amino acid 3700 to 3750, such as amino acid 3725 to 3775, for example amino acid 3750 to 3800, such as amino acid 3775 to 3825, for example amino acid 3800 to 3850, such as amino acid 3825 to 3875, for example amino acid 3850 to 3900, such as amino acid 3875 to 3925, for example amino acid 3900 to 3950, such as amino acid 3925 to 3975, for example amino acid 3950 to 4000, such as amino acid 3975 to 4025, amino acid 4001 to 4050, such as amino acid 4025 to 4075, for example amino acid 4050 to 4100, such as amino acid 4075 to 4125, for example amino acid 4100 to 4150, such as amino acid 4125 to 4175, for example amino acid 4150 to 4200, such as amino acid 4175 to 4225, for example amino acid 4200 to 4250, such as amino acid 4225 to 4275, for example amino acid 4250 to 4300, such as amino acid 4275 to 4325, for example amino acid 4300 to 4350, such as amino acid 4325 to 4375, for example amino acid 4350 to 4400, such as amino acid 4375 to 4425, for example amino acid 4400 to 4450, such as amino acid 4425 to 4475, for example amino acid 4450 to 4500, such as amino acid 4475 to 4525, for example amino acid 4500 to 4550, such as amino acid 4525 to 4575, for example amino acid 4550 to 4599 of the LRP1 B polypeptide sequence (SEQ ID NO: 4).

In one embodiment the steroid hormone binding protein receptor domain comprises fragments of the ApoE receptor 2 polypeptide sequence (SEQ ID NO: 5). For example such a fragment may comprise amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 963 of the ApoE receptor 2 polypeptide sequence (SEQ ID NO: 5).

In one embodiment the steroid hormone binding protein receptor domain essentially consists of or preferably consists of fragments of the ApoE receptor 2 polypeptide sequence (SEQ ID NO: 5). For example such a fragment may essentially consists of or preferably consists of amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino-acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 963 of the ApoE receptor 2 polypeptide sequence (SEQ ID NO: 5).

In another embodiment the steroid hormone binding protein receptor domain comprises fragments of the LRP polypeptide sequence (SEQ ID NO: 6). For example such a fragment may comprise amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 975, for example amino acid 950 to 1000, such as amino acid 975 to 1025, 1000 to 1050, such as amino acid 1025 to 1075, for example amino acid 1050 to 1100, such as amino acid 1075 to 1125, for example amino acid 1100 to 1150, such as amino acid 1125 to 1175, for example amino acid 1150 to 1200, such as amino acid 1175 to 1225, for example amino acid 1200 to 1250, such as amino acid 1225 to 1275, for example amino acid 1250 to 1300, such as amino acid 1275 to 1325, for example amino acid 1300 to 1350, such as amino acid 1325 to 1375, for example amino acid 1350 to 1400, such as amino acid 1375 to 1425, for example amino acid 1400 to 1450, such as amino acid 1425 to 1475, for example amino acid 1450 to 1500, such as amino acid 1475 to 1525, for example amino acid 1500 to 1550, such as amino acid 1525 to 1575, for example amino acid 1550 to 1600, such as amino acid 1575 to 1625, for example amino acid 1600 to 1650, such as amino acid 1625 to 1675, for example amino acid 1650 to 1700, such as amino acid 1675 to 1725, for example amino acid 1700 to 1750, such as amino acid 1725 to 1775, for example amino acid 1750 to 1800, such as amino acid 1775 to 1825, for example amino acid 1800 to 1850, such as amino acid 1825 to 1875, for example amino acid 1850 to 1900, such as amino acid 1875 to 1925, for example amino acid 1900 to 1950, such as amino acid 1925 to 1975, for example amino acid 1950 to 2000, such as amino acid 1975 to 2025, amino acid 2001 to 2050, such as amino acid 2025 to 2075, for example amino acid 2050 to 2100, such as amino acid 2075 to 2125, for example amino acid 2100 to 2150, such as amino acid 2125 to 2175, for example amino acid 2150 to 2200, such as amino acid 2175 to 2225, for example amino acid 2200 to 2250, such as amino acid 2225 to 2275, for example amino acid 2250 to 2300, such as amino acid 2275 to 2325, for example amino acid 2300 to 2350, such as amino acid 2325 to 2375, for example amino acid 2350 to 2400, such as amino acid 2375 to 2425, for example amino acid 2400 to 2450, such as amino acid 2425 to 2475, for example amino acid 2450 to 2500, such as amino acid 2475 to 2525, for example amino acid 2500 to 2550, such as amino acid 2525 to 2575, for example amino acid 2550 to 2600, such as amino acid 2575 to 2625, for example amino acid 2600 to 2650, such as amino acid 2625 to 2675, for example amino acid 650 to 700, such as amino acid 2675 to 2725, for example amino acid 2700 to 2750, such as amino acid 2725 to 2775, for example amino acid 2750 to 2800, such as amino acid 2775 to 2825, for example amino acid 2800 to 2850, such as amino acid 2825 to 2875, for example amino acid 2850 to 2900, such as amino acid 2875 to 2925, for example amino acid 2900 to 2950, such as amino acid 2925 to 2975, for example amino acid 2950 to 3000, such as amino acid 2975 to 3025, amino acid 3001 to 3050, such as amino acid 3025 to 3075, for example amino acid 3050 to 3100, such as amino acid 3075 to 3125, for example amino acid 3100 to 3150, such as amino acid 3125 to 3175, for example amino acid 3150 to 3200, such as amino acid 3175 to 3225, for example amino acid 3200 to 3250, such as amino acid 3225 to 3275, for example amino acid 3250 to 3300, such as amino acid 3275 to 3325, for example amino acid 3300 to 3350, such as amino acid 3325 to 3375, for example amino acid 3350 to 3400, such as amino acid 3375 to 3425, for example amino acid 3400 to 3450, such as amino acid 3425 to 3475, for example amino acid 3450 to 3500, such as amino acid 3475 to 3525, for example amino acid 3500 to 3550, such as amino acid 3525 to 3575, for example amino acid 3550 to 3600, such as amino acid 3575 to 3625, for example amino acid 3600 to 3650, such as amino acid 3625 to 3675, for example amino acid 3650 to 3700, such as amino acid 3675 to 3725, for example amino acid 3700 to 3750, such as amino acid 3725 to 3775, for example amino acid 3750 to 3800, such as amino acid 3775 to 3825, for example amino acid 3800 to 3850, such as amino acid 3825 to 3875, for example amino acid 3850 to 3900, such as amino acid 3875 to 3925, for example amino acid 3900 to 3950, such as amino acid 3925 to 3975, for example amino acid 3950 to 4000, such as amino acid 3975 to 4025, amino acid 4001 to 4050, such as amino acid 4025 to 4075, for example amino acid 4050 to 4100, such as amino acid 4075 to 4125, for example amino acid 4100 to 4150, such as amino acid 4125 to 4175, for example amino acid 4150 to 4200, such as amino acid 4175 to 4225, for example amino acid 4200 to 4250, such as amino acid 4225 to 4275, for example amino acid 4250 to 4300, such as amino acid 4275 to 4325, for example amino acid 4300 to 4350, such as amino acid 4325 to 4375, for example amino acid 4350 to 4400, such as amino acid 4375 to 4425, for example amino acid 4400 to 4450, such as amino acid 4425 to 4475, for example amino acid 4450 to 4500, such as amino acid 4475 to 4525, for example amino acid 4500 to 4550, such as amino acid 4525 to 4575, for example amino acid 4550 to 4599 of the LRP polypeptide sequence (SEQ ID NO: 6).

In another embodiment the steroid hormone binding protein receptor domain essentially consists of or preferably consists of fragments of the LRP polypeptide sequence (SEQ ID NO: 6). For example such a fragment may essentially consist of or preferably consist of amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 975, for example amino acid 950 to 1000, such as amino acid 975 to 1025, 1000 to 1050, such as amino acid 1025 to 1075, for example amino acid 1050 to 1100, such as amino acid 1075 to 1125, for example amino acid 1100 to 1150, such as amino acid 1125 to 1175, for example amino acid 1150 to 1200, such as amino acid 1175 to 1225, for example amino acid 1200 to 1250, such as amino acid 1225 to 1275, for example amino acid 1250 to 1300, such as amino acid 1275 to 1325, for example amino acid 1300 to 1350, such as amino acid 1325 to 1375, for example amino acid 1350 to 1400, such as amino acid 1375 to 1425, for example amino acid 1400 to 1450, such as amino acid 1425 to 1475, for example amino acid 1450 to 1500, such as amino acid 1475 to 1525, for example amino acid 1500 to 1550, such as amino acid 1525 to 1575, for example amino acid 1550 to 1600, such as amino acid 1575 to 1625, for example amino acid 1600 to 1650, such as amino acid 1625 to 1675, for example amino acid 1650 to 1700, such as amino acid 1675 to 1725, for example amino acid 1700 to 1750, such as amino acid 1725 to 1775, for example amino acid 1750 to 1800, such as amino acid 1775 to 1825, for example amino acid 1800 to 1850, such as amino acid 1825 to 1875, for example amino acid 1850 to 1900, such as amino acid 1875 to 1925, for example amino acid 1900 to 1950, such as amino acid 1925 to 1975, for example amino acid 1950 to 2000, such as amino acid 1975 to 2025, amino acid 2001 to 2050, such as amino acid 2025 to 2075, for example amino acid 2050 to 2100, such as amino acid 2075 to 2125, for example amino acid 2100 to 2150, such as amino acid 2125 to 2175, for example amino acid 2150 to 2200, such as amino acid 2175 to 2225, for example amino acid 2200 to 2250, such as amino acid 2225 to 2275, for example amino acid 2250 to 2300, such as amino acid 2275 to 2325, for example amino acid 2300 to 2350, such as amino acid 2325 to 2375, for example amino acid 2350 to 2400, such as amino acid 2375 to 2425, for example amino acid 2400 to 2450, such as amino acid 2425 to 2475, for example amino acid 2450 to 2500, such as amino acid 2475 to 2525, for example amino acid 2500 to 2550, such as amino acid 2525 to 2575, for example amino acid 2550 to 2600, such as amino acid 2575 to 2625, for example amino acid 2600 to 2650, such as amino acid 2625 to 2675, for example amino acid 2650 to 2700, such as amino acid 2675 to 2725, for example amino acid 2700 to 2750, such as amino acid 2725 to 2775, for example amino acid 2750 to 2800, such as amino acid 2775 to 2825, for example amino acid 2800 to 2850, such as amino acid 2825 to 2875, for example amino acid 2850 to 2900, such as amino acid 2875 to 2925, for example amino acid 2900 to 2950, such as amino acid 2925 to 2975, for example amino acid 2950 to 3000, such as amino acid 2975 to 3025, amino acid 3001 to 3050, such as amino acid 3025 to 3075, for example amino acid 3050 to 3100, such as amino acid 3075 to 3125, for example amino acid 3100 to 3150, such as amino acid 3125 to 3175, for example amino acid 3150 to 3200, such as amino acid 3175 to 3225, for example amino acid 3200 to 3250, such as amino acid 3225 to 3275, for example amino acid 3250 to 3300, such as amino acid 3275 to 3325, for example amino acid 3300 to 3350, such as amino acid 3325 to 3375, for example amino acid 3350 to 3400, such as amino acid 3375 to 3425, for example amino acid 3400 to 3450, such as amino acid 3425 to 3475, for example amino acid 3450 to 3500, such as amino acid 3475 to 3525, for example amino acid 3500 to 3550, such as amino acid 3525 to 3575, for example amino acid 3550 to 3600, such as amino acid 3575 to 3625, for example amino acid 3600 to 3650, such as amino acid 3625 to 3675, for example amino acid 3650 to 3700, such as amino acid 3675 to 3725, for example amino acid 3700 to 3750, such as amino acid 3725 to 3775, for example amino acid 3750 to 3800, such as amino acid 3775 to 3825, for example amino acid 3800 to 3850, such as amino acid 3825 to 3875, for example amino acid 3850 to 3900, such as amino acid 3875 to 3925, for example amino acid 3900 to 3950, such as amino acid 3925 to 3975, for example amino acid 3950 to 4000, such as amino acid 3975 to 4025, amino acid 4001 to 4050, such as amino acid 4025 to 4075, for example amino acid 4050 to 4100, such as amino acid 4075 to 4125, for example amino acid 4100 to 4150, such as amino acid 4125 to 4175, for example amino acid 4150 to 4200, such as amino acid 4175 to 4225, for example amino acid 4200 to 4250, such as amino acid 4225 to 4275, for example amino acid 4250 to 4300, such as amino acid 4275 to 4325, for example amino acid 4300 to 4350, such as amino acid 4325 to 4375, for example amino acid 4350 to 4400, such as amino acid 4375 to 4425, for example amino acid 4400 to 4450, such as amino acid 4425 to 4475, for example amino acid 4450 to 4500, such as amino acid 4475 to 4525, for example amino acid 4500 to 4550, such as amino acid 4525 to 4575, for example amino acid 4550 to 4599 of the LRP polypeptide sequence (SEQ ID NO: 6).

In another embodiment the steroid hormone binding protein receptor domain comprises fragments of VLDL receptor polypeptide sequence (SEQ ID NO: 7). For example such a fragment may comprise amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 873 of the VLDL receptor polypeptide sequence (SEQ ID NO: 7).

In another embodiment the steroid hormone binding protein receptor domain essentially consists of or preferably consists of fragments of the VLDL receptor polypeptide sequence (SEQ ID NO: 7). For example such a fragment may essentially consist of or preferably consist of amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 873 of the VLDL receptor polypeptide sequence (SEQ ID NO: 7).

Preferably, the steroid hormone binding protein receptor domain is selected from the group consisting of fragments of the ApoE receptor 2 polypeptide sequence (SEQ ID NO: 5), fragments of the megalin polypeptide sequence (SEQ ID NO: 3) and fragments of the VLDL receptor polypeptide sequence (SEQ ID NO: 7) as outlined above.

The compound according to the present invention may also be a polypeptide, wherein said polypeptide is a domain of a steroid hormone binding protein co-receptor. Preferably, such a domain is capable of associating with either a steroid hormone binding protein and/or a steroid hormone binding protein receptor. More preferably, the domain only associates with a steroid hormone binding protein or a steroid hormone binding protein receptor, but not both. Accordingly, such a polypeptide could competitively inhibit the association between a steroid hormone binding protein and a steroid hormone binding protein co-receptor or the association between a steroid hormone binding protein co-receptor and a steroid hormone binding protein receptor, which would result in decreased uptake of steroid hormone into cells.

In one embodiment the steroid hormone binding protein co-receptor domain comprises fragments of the cubilin polypeptide sequence (SEQ ID NO: 8). For example such a fragment may comprise amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 975, for example amino acid 950 to 1000, such as amino acid 975 to 1025, 1000 to 1050, such as amino acid 1025 to 1075, for example amino acid 1050 to 1100, such as amino acid 1075 to 1125, for example amino acid 1100 to 1150, such as amino acid 1125 to 1175, for example amino acid 1150 to 1200, such as amino acid 1175 to 1225, for example amino acid 1200 to 1250, such as amino acid 1225 to 1275, for example amino acid 1250 to 1300, such as amino acid 1275 to 1325, for example amino acid 1300 to 1350 such as amino acid 1325 to 1375, for example amino acid 1350 to 1400, such as amino acid 1375 to 1425, for example amino acid 1400 to 1450, such as amino acid 1425 to 1475, for example amino acid 1450 to 1500, such as amino acid 1475 to 1525, for example amino acid 1500 to 1550, such as amino acid 1525 to 1575, for example amino acid 1550 to 1600, such as amino acid 1575 to 1625, for example amino acid 1600 to 1650, such as amino acid 1625 to 1675, for example amino acid 1650 to 1700, such as amino acid 1675 to 1725, for example amino acid 1700 to 1750, such as amino acid 1725 to 1775, for example amino acid 1750 to 1800, such as amino acid 1775 to 1825, for example amino acid 1800 to 1850, such as amino acid 1825 to 1875, for example amino acid 1850 to 1900, such as amino acid 1875 to 1925, for example amino acid-1900 to 1950, such as amino acid 1925 to 1975, for example amino acid 1950 to 2000, such as amino acid 1975 to 2025, amino acid 2001 to 2050, such as amino acid 2025 to 2075, for example amino acid 2050 to 2100, such as amino acid 2075 to 2125, for example amino acid 2100 to 2150, such as amino acid 2125 to 2175, for example amino acid 2150 to 2200, such as amino acid 2175 to 2225, for example amino acid 2200 to 2250, such as amino acid 2225 to 2275, for example amino acid 2250 to 2300, such as amino acid 2275 to 2325, for example amino acid 2300 to 2350, such as amino acid 2325 to 2375, for example amino acid 2350 to 2400, such as amino acid 2375 to 2425, for example amino acid 2400 to 2450, such as amino acid 2425 to 2475, for example amino acid 2450 to 2500, such as amino acid 2475 to 2525, for example amino acid 2500 to 2550, such as amino acid 2525 to 2575, for example amino acid 2550 to 2600, such as amino acid 2575 to 2625, for example amino acid 2600 to 2650, such as amino acid 2625 to 2675, for example amino acid 650 to 700, such as amino acid 2675 to 2725, for example amino acid 2700 to 2750, such as amino acid 2725 to 2775, for example amino acid 2750 to 2800, such as amino acid 2775 to 2825, for example amino acid 2800 to 2850, such as amino acid 2825 to 2887 of the cubilin polypeptide sequence (SEQ ID NO: 8).

In another embodiment the steroid hormone binding protein co-receptor domain essentially consists of or preferably consists of fragments of the cubilin polypeptide sequence (SEQ ID NO: 8). For example such a fragment may essentially consist of or preferably consist of amino acid 1 to 50, such as amino acid 25 to 75, for example amino acid 50 to 100, such as amino acid 75 to 125, for example amino acid 100 to 150, such as amino acid 125 to 175, for example amino acid 150 to 200, such as amino acid 175 to 225, for example amino acid 200 to 250, such as amino acid 225 to 275, for example amino acid 250 to 300, such as amino acid 275 to 325, for example amino acid 300 to 350, such as amino acid 325 to 375, for example amino acid 350 to 400, such as amino acid 375 to 425, for example amino acid 400 to 450, such as amino acid 425 to 475, for example amino acid 450 to 500, such as amino acid 475 to 525, for example amino acid 500 to 550, such as amino acid 525 to 575, for example amino acid 550 to 600, such as amino acid 575 to 625, for example amino acid 600 to 650, such as amino acid 625 to 675, for example amino acid 650 to 700, such as amino acid 675 to 725, for example amino acid 700 to 750, such as amino acid 725 to 775, for example amino acid 750 to 800, such as amino acid 775 to 825, for example amino acid 800 to 850, such as amino acid 825 to 875, for example amino acid 850 to 900, such as amino acid 875 to 925, for example amino acid 900 to 950, such as amino acid 925 to 975, for example amino acid 950 to 1000, such as amino acid 975 to 1025, 1000 to 1050, such as amino acid 1025 to 1075, for example amino acid 1050 to 1100, such as amino acid 1075 to 1125, for example amino acid 1100 to 1150, such as amino acid 1125 to 1175, for example amino acid 1150 to 1200, such as amino acid 1175 to 1225, for example amino acid 1200 to 1250, such as amino acid 1225 to 1275, for example amino acid 1250 to 1300, such as amino acid 1275 to 1325, for example amino acid 1300 to 1350, such as amino acid 1325 to 1375, for example amino acid 1350 to 1400, such as amino acid 1375 to 1425, for example amino acid 1400 to 1450, such as amino acid 1425 to 1475, for example amino acid 1450 to 1500, such as amino acid 1475 to 1525, for example amino acid 1500 to 1550, such as amino acid 1525 to 1575, for example amino acid 1550 to 1600, such as amino acid 1575 to 1625, for example amino acid 1600 to 1650, such as amino acid 1625 to 1675, for example amino acid 1650 to 1700, such as amino acid 1675 to 1725, for example amino acid 1700 to 1750, such as amino acid 1725 to 1775, for example amino acid 1750 to 1800, such as amino acid 1775 to 1825, for example amino acid 1800 to 1850, such as amino acid 1825 to 1875, for example amino acid 1850 to 1900, such as amino acid 1875 to 1925, for example amino acid 1900 to 1950, such as amino acid 1925 to 1975, for example amino acid 1950 to 2000, such as amino acid 1975 to 2025, amino acid 2001 to 2050, such as amino acid 2025 to 2075, for example amino acid 2050 to 2100, such as amino acid 2075 to 2125, for example amino acid 2100 to 2150, such as-amino acid 2125 to 2175, for example amino acid 2150 to 2200, such as amino acid 2175 to 2225, for example amino acid 2200 to 2250, such as amino acid 2225 to 2275, for example amino acid 2250 to 2300, such as amino acid 2275 to 2325, for example amino acid 2300 to 2350, such as amino acid 2325 to 2375, for example amino acid 2350 to 2400, such as amino acid 2375 to 2425, for example amino acid 2400 to 2450, such as amino acid 2425 to 2475, for example amino acid 2450 to 2500, such as amino acid 2475 to 2525, for example amino acid 2500 to 2550, such as amino acid 2525 to 2575, for example amino acid 2550 to 2600, such as amino acid 2575 to 2625, for example amino acid 2600 to 2650, such as amino acid 2625 to 2675, for example amino acid 2650 to 2700, such as amino acid 2675 to 2725, for example amino acid 2700 to 2750, such as amino acid 2725 to 2775, for example amino acid 2750 to 2800, such as amino acid 2775 to 2825, for example amino acid 2800 to 2850, such as amino acid 2825 to 2887 of the cubilin polypeptide sequence (SEQ ID NO: 8).

In another embodiment of the invention the polypeptide is a steroid hormone binding protein, for example SHBG.

In yet another preferred embodiment the polypeptide is a fragment of a steroid hormone binding protein. Preferably, such a fragment is capable of associating with a steroid hormone binding protein receptor or a with a steroid hormone binding protein co-receptor, more preferably, such a fragment is furthermore not capable of associating with a steroid hormone, i.e. the fragment can bind the steroid hormone binding protein receptor or the steroid hormone binding protein co-receptor and thereby inhibit binding of a steroid hormone binding protein/steroid hormone complex to said receptor/co-receptor.

In one embodiment of the present invention the steroid hormone binding protein domain is capable of associating with a steroid hormone binding protein receptor domain, preferably the steroid hormone binding protein domain is capable of associating a steroid hormone binding protein receptor domain comprising at least one complement-type repeat or even more preferably consisting of at least one complement-type repeat, such as one, for example 2, such as 3, for example 4, such as 5, for example 6, such as 7, for example 8, such as 9, for example 10, such as more than 10 complement-type repeats. The domain structure of selected examples of steroid hormone binding protein receptors is given in FIG. 6.

In one embodiment such a fragment comprise at least amino acid 47 to 167 of human SHBG (SEQ ID NO: 1), however more preferably the fragment comprise additionally 1 amino acid, such as at least 2 amino acids, for example at least 5 amino acids, such as at least 10 amino acid at the C-terminus and comprise additionally 1 amino acid, such as at least 2 amino acids, for example at least 5 amino acids, such as at least 10 amino acid at the N-terminus.

In another embodiment the fragment comprise amino acid 128-137 of human SHBG (SEQ ID NO: 1) and in yet another embodiment the fragment comprise amino acid 106 to amino acid 125 of human SHBG (SEQ ID NO: 1).

In one embodiment such a fragment consists of amino acid 47 to 167 of human SHBG (SEQ ID NO: 1), or for example the fragment consists of amino acid 128-137 of human SHBG (SEQ ID NO: 1) or the fragment consists of amino acid 106 to amino acid 125 of human SHBG (SEQ ID NO: 1).

Functional homologues of the above mentioned fragments of steroid hormone binding proteins are also within the scope of the present invention: Functional homologues are described in more detail herein below. It should be noted however, that fragments of SHBG as outlined herein above comprise the basic clusters:

Arg 47, Arg 166 and Arg 167

Lys 106, Arg 123 and Arg 125

and functional homologues preferably should be selected such as these basic clusters are maintained. Accordingly, the amino acid corresponding to: position 47 in the full length protein (SEQ ID NO: 1) is preferably selected from the group consisting of Arg and Lys, position 106 in the full length protein (SEQ ID NO: 1) is preferably selected from the group consisting of Arg and Lys, position 123 in the full length protein (SEQ ID NO: 1) is preferably selected from the group consisting of Arg and Lys, position 125 in the full length protein (SEQ ID NO: 1) is preferably selected from the group consisting of Arg and Lys, position 166 in the full length protein (SEQ ID NO: 1) is preferably selected from the group consisting of Arg and Lys, position 167 in the full length protein is preferably selected from the group consisting of Arg and Lys.

The compound according to the present invention could also be selected from the group comprising fragments of RAP that can associate with a steroid hormone binding protein receptor.

In one additional embodiment of the present invention, the compound is a nucleic acid sequence. Preferably, such a nucleic acid sequence potentially alters the expression of a steroid hormone binding protein receptor or a steroid hormone binding protein co-receptor.

In one preferred embodiment such a nucleic acid sequence comprise a DNA sequence encoding for an antisense RNA or a small interfering RNA (siRNA) of a steroid hormone binding protein receptor or the nucleic acid sequence is an antisense RNA of a steroid hormone binding protein receptor. Homologues thereof are also within the scope of the present invention. Alternatively, such a nucleic acid sequence could comprise a DNA sequence encoding for an antisense RNA of a steroid hormone binding protein co-receptor or the nucleic acid sequence is an antisense RNA of a steroid hormone binding protein co-receptor or homologues thereof.

Furthermore, the nucleic acid sequence may comprise an-antigene nucleic acid sequence, which is capable of hybridising with a gene encoding a steroid hormone binding protein receptor or a steroid hormone binding protein co-receptor and thereby inhibiting transcription of said gene. Said antigene nucleic acid sequence may be capable of hybridising to any part of said gene, for example to the promotor and/or to introns and/or to exons of sid gene. The antigene nucleic acid may be any kind of nucleic acid, for example DNA, RNA, LNA or PNA or siRNA.

In the context of the present invention the term “antisense RNA” is intended to encompass an RNA sequence transcribed from the non-coding DNA strand of a steroid hormone binding protein receptor or a steroid hormone binding protein co-receptor gene or an RNA sequence that is capable of hybridising to a steroid hormone binding protein receptor or a steroid hormone binding protein co-receptor mRNA under stringent conditions or fragments thereof.

Preferably, said antisense nucleic acid is capable of hybridising to a nucleic acid encoding a polypeptide as defined in any of SEQ ID 2, 3, 4, 5, 6, 7 or 8 or parts thereof under stringent conditions (see definition of stringent conditions herein below).

If the nucleic acid sequence is a DNA sequence encoding an antisense RNA of a steroid hormone binding protein receptor or steroid hormone binding protein co-receptor or homologues thereof, such a nucleotide sequence is preferably operably linked to nucleotide sequences that directs transcription of said DNA sequence in the cell of the particular embodiment of the invention.

In another embodiment the nucleic acid sequence comprises sequences encoding a steroid hormone binding protein receptor or steroid hormone binding protein co-receptor or homologues thereof or fragments thereof. Such a nucleic acid sequence is preferably operably linked to nucleotide sequences that directs transcription of said DNA sequence in the cell of the particular embodiment of the invention.

A variety of nucleotide sequences that directs transcription of DNA sequences are known to the person skilled in the art and such sequences should be selected according to the specific need in the individual case. For example such sequences could be promoter sequences and enhancer sequences of prokaryotic, eukaryotic or viral origin or they could be synthetic sequences.

The nucleic acid sequence may be comprised within a vector and any suitable vector known to the person skilled in the art may be employed with the present invention. A vector is cable of delivering the nucleic acid molecule into a host cell. Such a vector contains nucleic acid sequences that are not naturally found adjacent to the nucleic acid sequences of the present invention.

A vector is a replicable construct which could be any nucleic acid including DNA, RNA, LNA and PNA. Once transformed into a suitable host, the vector replicates and functions independently of the host genome, or may, in some instances, integrate into the genome itself.

Typically the vector is a viral derived vector, a retroviral derived vector, a phage, a plasmid, a cosmid, an integratable DNA fragment (i.e., integratable into the host genome by recombination), bacteria or eukaryotic cells.

Functional Homologues

Functional homologues of polypeptides according to the present invention is meant to comprise any polypeptide sequence which is capable of associating with a steroid hormone binding protein and/or steroid hormone binding protein receptor and/or steroid hormone binding protein co-receptor and thereby prevents association between the steroid hormone binding protein and/or the steroid hormone binding protein receptor or steroid hormone binding protein co-receptor.

Functional homologues according to the present invention comprise polypeptides with an amino acid sequence, which are sharing at least some homology with the predetermined polypeptide sequences as outlined herein above. For example such polypeptides are at least about 40 percent, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous with the predetermined polypeptide sequences as outlined herein above.

The homology between amino acid sequences may be calculated using well known algorithms such as for example any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

Functional homologues may comprise an amino acid sequence that comprises at least one substitution of one amino acid for any other amino acid. For example such a substitution may be a conservative amino acid substitution or it may be a non-conservative substitution.

A conservative amino acid substitution is a substitution of one amino acid within a predetermined group of amino acids for another amino acid within the same group, wherein the amino acids within predetermined groups exhibit similar or substantially similar characteristics. Within the meaning of the term “conservative amino acid substitution” as applied herein, one amino acid may be substituted for another within groups of amino acids characterised by having

i) polar side chains (Asp, Glu, Lys, Arg, His, Asn, Gin, Ser, Thr, Tyr, and Cys,)

ii) non-polar side chains (Gly, Ala, Val, Leu, lie, Phe, Trp, Pro, and Met)

iii) aliphatic side chains (Gly, Ala Val, Leu, lie)

iv) cyclic side chains (Phe, Tyr, Trp, His, Pro)

v) aromatic side chains (Phe, Tyr, Trp)

vi) acidic side chains (Asp, Glu)

vii) basic side chains (Lys, Arg, His)

viii) amide side chains (Asn, Gln)

ix) hydroxy side chains (Ser, Thr)

x) sulphor-containing side chains (Cys, Met), and

xi) amino acids being monoamino-dicarboxylic acids or monoamino-monocarboxylic-monoamidocarboxylic acids (Asp, Glu, Asn, Gin).

Non-conservative substitutions are any other substitutions. A non-conservative substitution leading to the formation of a functional homologue would for example i) differ substantially in hydrophobicity, for example a hydrophobic residue (Val, Ile, Leu, Phe or Met) substituted for a hydrophilic residue such as Arg, Lys, Trp or Asn, or a hydrophilic residue such as Thr, Ser, His, Gln, Asn, Lys, Asp, Glu or Trp substituted for a hydrophobic residue; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Functional homologues according to the present invention may comprise more than one such substitution, such as e.g. two amino acid substitutions, for example three or four amino acid substitutions, such as five or six amino acid substitutions, for example seven or eight amino acid substitutions, such as from 10 to 15 amino acid substitutions, for example from 15 to 25 amino acid substitution, such as from 25 to 30 amino acid substitutions, for example from 30 to 40 amino acid substitution, such as from 40 to 50 amino acid substitutions, for example from 50 to 75 amino acid substitution, such as from 75 to 100 amino acid substitutions, for example more than 100 amino acid substitutions.

The addition or deletion of an amino acid may be an addition or deletion of from 2 to 5 amino acids, such as from 5 to 10 amino acids, for example from 10 to 20 amino acids, such as from 20 to 50 amino acids. However, additions or deletions of more than 50 amino acids, such as additions from 50 to 200 amino acids, are also comprised within the present invention.

The polypeptides according to the present invention, including any variants and functional homologues thereof, may in one embodiment comprise more than 5 amino acid residues, such as more than 10 amino acid residues, for example more than 20 amino acid residues, such as more than 25 amino acid residues, for example more than 50 amino acid residues, such as more than 75 amino acid residues, for example more than 100 amino acid residues, such as more than 150 amino acid residues, for example more than 200 amino acid residues.

Additional factors may be taken into consideration when determining functional homologues according to the meaning used herein. For example functional homologues may be capable of associating with antisera which are specific for the polypeptides according to the present invention.

In a further embodiment the present invention relates to functional equivalents which comprise substituted amino acids having hydrophilic or hydropathic indices that are within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted.

The importance of the hydrophilic and hydropathic amino acid indices in conferring interactive biologic function on a protein is well understood in the art (Kyte & Doolittle, 1982 and Hopp, U.S. Pat. No. 4,554,101, each incorporated herein by reference).

The amino acid hydropathic index values as used herein are: isoleucine (+4.5); valine (+4.2); leucine (+3.8); phenylalanine (+2.8); cysteine/cystine (+2.5); rnethionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (4.5) (Kyte & Doolittle, 1982).

The amino acid hydrophilicity values are: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5 .+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) (U.S. Pat. No. 4,554,101).

Substitution of amino acids can therefore in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In addition to the polypeptide compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates, including dimers or unrelated chemical moieties. Such functional equivalents are prepared by linkage of functionalities to groups which are found in fragment including at any one or both of the N- and C-termini, by means known in the art.

Functional equivalents may thus comprise fragments conjugated to aliphatic or acyl esters or amides of the carboxyl terminus, alkylamines or residues containing carboxyl side chains, e.g., conjugates to alkylamines at aspartic acid residues; O-acyl derivatives of hydroxyl group-containing residues and N-acyl derivatives of the amino terminal amino acid or amino-group containing residues, e.g. conjugates with Met-Leu-Phe. Derivatives of the acyl groups are selected from the group of alkyl-moieties (including C3 to C10 normal alkyl), thereby forming alkanoyl species, and carbocyclic or heterocyclic compounds, thereby forming aroyl species. The reactive groups preferably are difunctional compounds known per se for use in cross-linking proteins to insoluble matrices through reactive side groups.

Homologues of nucleic acid sequences within the scope of the present invention are nucleic acid sequences, which encodes an RNA and/or a protein with similar biological function, and which is either

a) at least 50% identical, such as at least 60% identical, for example at least 70% identical, such as at least 75% identical, for example at least 80% identical, such as at least 85% identical, for example at least 90% identical, such as at least 95% identical

b) or able to hybridise to the complementary strand of said nucleic acid sequence under stringent conditions.

Stringent conditions as used herein shall denote stringency as normally applied in connection with Southern blotting and hybridisation as described e.g. by Southern E. M., 1975, J. Mol. Biol. 98:503-517. For such purposes it is routine practise to include steps of prehybridization and hybridization. Such steps are normally performed using solutions containing 6×SSPE, 5% Denhardt's, 0.5% SDS, 50% formamide, 100 μg/ml denaturated salmon testis DNA (incubation for 18 hrs at 42° C.), followed by washings with 2×SSC and 0.5% SDS (at room temperature and at 37° C.), and a washing with 0.1×SSC and 0.5% SDS (incubation at 68° C. for 30 min), as described by Sambrook et al., 1989, in “Molecular Cloning/A Laboratory Manual”, Cold Spring Harbor), which is incorporated herein by reference.

Homologous of nucleic acid sequences also encompass nucleic acid sequences which comprise additions and/or deletions. Such additions and/or deletions may be internal or at the end. Additions and/or deletions may be of 1-5 nucleotides, such as 5 to 10 nucleotide, for example 10 to 50 nucleotides, such as 50 to 100 nucleotides, for example at least 100 nucleotides.

Medical Use

A number of clinical conditions may be treated by functional inhibition or functional activation of steroid hormones. Accordingly, the compounds according to the invention that alter the uptake of steroid hormones into cells presenting a steroid hormone binding receptor may be useful in treatment of said clinical conditions.

Hence, it is an object of the present invention to provide use of a compound according to the invention for the preparation of a pharmaceutical composition for the treatment of a clinical condition associated with a steroid hormone.

The clinical conditions associated with a steroid hormone may for example be a condition characterised by dependency on one or more steroid hormones or by insufficiency of one or more steroid hormones.

Hence, in one embodiment of the present invention the clinical condition may be characterised by the undesirable presence and/or growth of cells dependent on one or more steroid hormones. For example, the cells of certain tumours are dependent on steroid hormones for growth.

The clinical condition may for example be selected from the group consisting of prostate cancer and breast cancer. In the prior art several strategies have been used to functional inhibit steroid hormone function in prostate or breast cancer. An overview of the various mechanisms of antagonists in steroid dependent tumour therapy is given in FIG. 10.

The compounds according to the present invention useful in tumour therapy are in particular inhibitors of steroid hormone uptake into cells. Because said inhibitors primarily inhibit active receptor mediated or receptor/co-receptor mediated uptake of steroid hormones into cells, they will in general not inhibit diffusion of steroid hormones into cells. Accordingly, primarily tissues depending on large amounts of steroid hormones through uptake via receptors will be affected by the treatment.

Compounds useful for treatment of breast cancer in particular include compounds that inhibit or reduce cellular uptake of estrogens, such as estradiols, for example 17β-estradiol.

Compounds useful for treatment of prostate cancer in particular include compounds that inhibit or reduce cellular uptake of androgens, such as testosterone. In one embodiment said compounds are capable of inhibiting uptake of androgens, by inhibiting uptake of steroid hormone/steroid hormone binding protein complexes by megalin and/or the ApoER2.

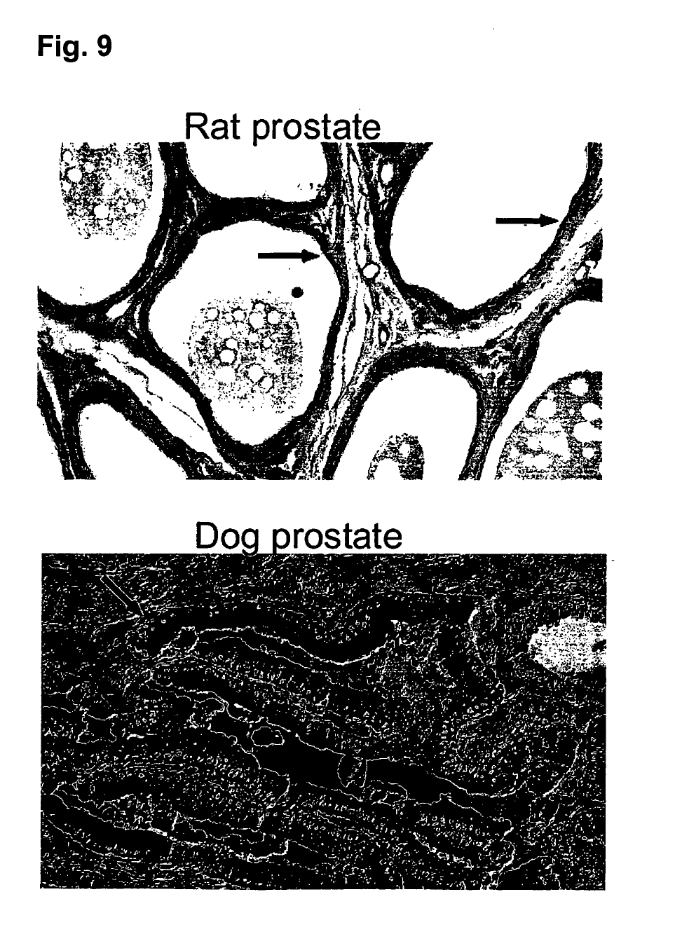

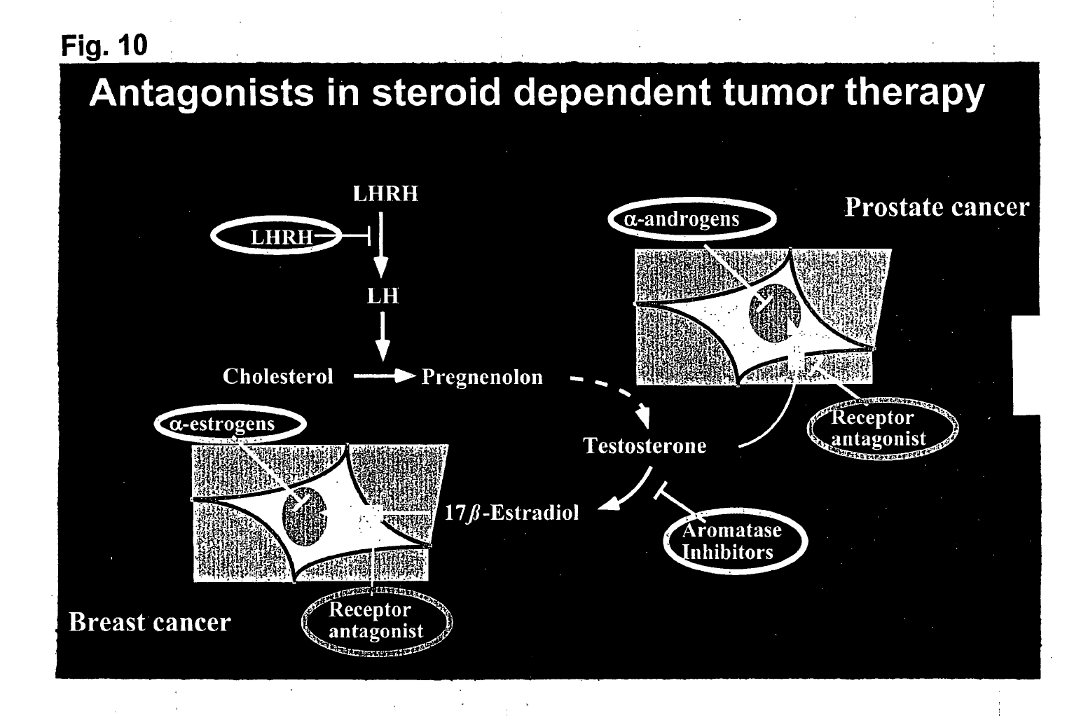

Cells of the prostate of several mammalian species expresses steroid hormone binding protein receptors. For example epithelial cells of dog and rat prostate expresses megalin (see example 5 and FIG. 9). Accordingly, it is preferred that compounds for treatment of prostate cancer inhibit or reduce cellular uptake of androgens, such as testosterone complexed to an androgen binding protein such as SHBG by the megalin receptor.

The compounds according to the present invention that may be used to treat prostate cancer or breast cancer, may be used alone or in combination with one or more other therapies against cancer. Such therapies include but are not limited to surgery, chemotherapy, radiotherapy, gene therapy, therapy with cytokines and immunotherapy.

In one preferred embodiment the compounds of the invention may be administered either simultaneously or sequentially in any order in combination with one or more other steroid hormone antagonist. Said antagonist may for example be any of the antagonist mentioned in FIG. 10. For breast cancer therapy, the antagonist may for example be selected from the group consisting of LHRH, aromatase inhibitors, estrogen receptor antagonists and α-estrogens. For prostate cancer therapy, the antagonist may for example be selected from the group consisting of LHRH, androgen receptor antagonists and α-androgens.

Contraception

In one aspect the present invention relates to contraceptive compositions comprising one or more compounds according to the invention. The present invention also relates to methods of preventing pregnancy comprising administering a sufficient amount of a compound, which alters the uptake of steroid hormone into cells presenting a steroid hormone binding protein receptor. Furthermore the invention provides use of such a compound for the preparation of a composition that may be useful to prevent pregnancy.

In the male genital tract, SHBG is produced by Sertoli cells in the testis and secreted into the lumen of the seminiferous tubules [Feldman et al, 1981, French et al., 1973]. There it binds testosterone, present in large amounts in the testicular fluids. Via the efferent ducts, SHBG is transported to the epididymis and internalised by principal cells lining the epididymal duct [Feldman et al., 1981, Gerard et al., 1988]. Because the principal cells in the epididymis are responsible for the conversion of testosterone into 5α-dihydrotestosterone (DHT) that is required for sperm maturation, endocytic uptake of SHBG may be a route to deliver testosterone to these cells. Binding studies indicate that the receptor binds SHBG with a Kd of 50 nM when assayed in membrane suspensions, and a Kd of 0.5 μM in solubilized membranes [Fraira et al., 1991].

Steroid hormone binding protein receptors are expressed in the epididymis, for example megalin and ApoER2 are expressed in the epididymis.

Hence, sperm maturation in the epididymis may be dependent on uptake of testosterone into the principal cells. It is therefore an object of the present invention to provide a contraceptive composition, comprising a compound capable of altering the uptake of a steroid hormone into cells of the epididymis. More preferably, said compound is capable of inhibiting or reducing the uptake of an androgen into cells of the epididymis, even more preferably, said compound is capable of inhibiting or reducing the uptake of testosterone into the principal cells of the epidymis.

Such contraceptive compositions are in particular useful for male contraception.

Furthermore, the present invention provides methods of preventing pregnancy comprising administering a sufficient amount of a compound capable of altering the uptake of a steroid hormone into cells of the epididymis. More preferably, said compound is capable of inhibiting or reducing the uptake of an androgen into cells of the epididymis, even more preferably, said compound is capable of inhibiting or reducing the uptake of testosterone into the principal cells of the epidymis.

Pharmaceutical Compositions

The individual to receive treatment is any animal, however, preferably the individual is a human being.

In the embodiments of the present invention providing methods of preventing pregnancy, the individual is preferably a male individual.

The main routes of drug delivery according to the present invention are intravenous, oral, and topical, as will be described below. Other drug-administration methods, such as subcutaneous injection, which are effective to deliver the drug to a target site or to introduce the drug into the bloodstream, are also contemplated.

The mucosal membrane to which the pharmaceutical preparation of the invention is administered may be any mucosal membrane of the mammal to which the biologically active substance is to be given, e.g. in the nose, vagina, eye, mouth, genital tract, lungs, gastrointestinal tract, or rectum.

Compounds of the invention may be administered parenterally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques. The compounds may also be administered by inhalation, that is by intranasal and oral inhalation administration.

The compounds according to the invention may be administered with at least one other compound. The compounds may be administered simultaneously, either as separate formulations or combined in a unit dosage form, or administered sequentially.

The dosage requirements will vary with the particular drug composition employed, the route of administration and the particular individual being treated. Ideally, an individual to be treated by the present method will receive a pharmaceutically effective amount of the compound in the maximum tolerated dose; generally no higher than that required before drug resistance develops.

For all methods of use disclosed herein for the compounds, the daily oral dosage regimen will preferably be from about 0.01 to about 80 mg/kg of total body weight. The daily parenteral dosage regimen about 0.001 to about 80 mg/kg of total body weight. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day. It will also be recognised by one of skill in the art that the optimal quantity and spacing of individual dosages of a compound or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound or a pharmaceutically acceptable salt thereof given per day for a defined number of days, can be ascertained by those skilled in the art using conventional course of treatment determination tests.

The term “unit dosage form” as used herein refers to physically discrete units suitable as unitary dosages for human and animal individuals, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on interindividual differences in pharmacokinetics, drug distribution, and metabolism. The “effective level” can be defined, for example, as the blood or tissue level desired in the individual that corresponds to a concentration of one or more compounds according to the invention.

Pharmaceutical compositions containing a compound of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications.

Pharmaceutical acceptable salts of the compounds according to the present invention should also be considered to fall within the scope of the present invention. Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

Whilst it is possible for the compounds or salts of the present invention to be administered as the raw chemical, it is preferred to present them in the form of a pharmaceutical formulation. Accordingly, the present invention further provides a pharmaceutical formulation, for medicinal application, which comprises a compound of the present invention or a pharmaceutically acceptable salt thereof, as herein defined, and a pharmaceutically acceptable carrier therefor.

The compounds of the present invention may be formulated in a wide variety of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilisers, lubricants, suspending agents, binders, preservatives, wetting agents, tablet disintegrating agents, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably containing from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilised by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations, which are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavours, stabilising and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilisers, buffers, artificial and natural sweeteners, dispersants, thickeners, solubilising agents, and the like.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilising and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammbnium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimise or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The compounds of the invention can also be delivered topically. Regions for topical administration include the skin surface and also mucous membrane tissues of the vagina, rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, for example. Methods for preparing such compositions are well known in the pharmaceutical industry.

The compounds of the present invention may be formulated for topical administration to the epidermis as ointments, creams or lotions, or as a transdermal patch. Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturiser such as glycerol or an oil such as castor oil or arachis oil.

The pharmaceutical active compound described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

Transdermal delivery is accomplished by exposing a source of the active compound to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Bemer (eds.), CRC Press, (1987). Such dosage forms can be made by dissolving, dispersing, or otherwise incorporating the pharmaceutical active compound in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

The compounds of the present invention may be formulated for vaginal administration. Pessaries, tampons, creams, gels, pastes, foams or sprays containing in addition to the active ingredient such carriers as are known in the art to be appropriate.

When desired, formulations can be prepared with enteric coatings adapted for sustained or controlled release administration of the active ingredient.

Pharmaceutical compositions usually comprise a carrier. Illustrative solid carrier include lactose, terra alba, sucrose, talc, gelatin, agar, pectin, acacia, magnesium stearate, stearic acid and the like. A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions, and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyviriylpyrrolidine, low melting waxes and ion exchange resins.

Illustrative liquid carriers include syrup, peanut oil, olive oil, water, etc. Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilisers, emulsifiers, buffers, preservatives, sweeteners, flavouring agents, suspending agents, thickening agents, colours, viscosity regulators, stabilisers or osmo-regulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carders are useful in sterile liquid form compositions for parenteral administration. The liquid carrier for pressurised compositions can be halogenated hydrocarbon or other pharmaceutically acceptable propellant. Liquid pharmaceutical compositions which are sterile solutions or suspensions can be utilised by, for example, intramuscular, intraperitoneal or subcutaneous injection. Sterile solutions can also be administered intravenously. The compound can also be administered orally either in liquid or solid composition form.

The carrier or excipient may include time delay material well known to the art, such as glyceryl monostearate or glyceryl distearate along or with a wax, ethylcellulose, hydroxypropylmethylcellulose, methylmethacrylate and the like. When formulated for oral administration, 0.01% Tween 80 in PHOSAL PG-50 (phospholipid concentrate with 1,2-propylene glycol, A. Nattermann & Cie. GmbH) has been recognised as providing an acceptable oral formulation for other compounds, and may be adapted to formulations for various compounds of this invention.

### EXAMPLES

### Example 1

Surface-plasmon resonance analysis of binding of testosterone/SHBG complexes to purified megalin.

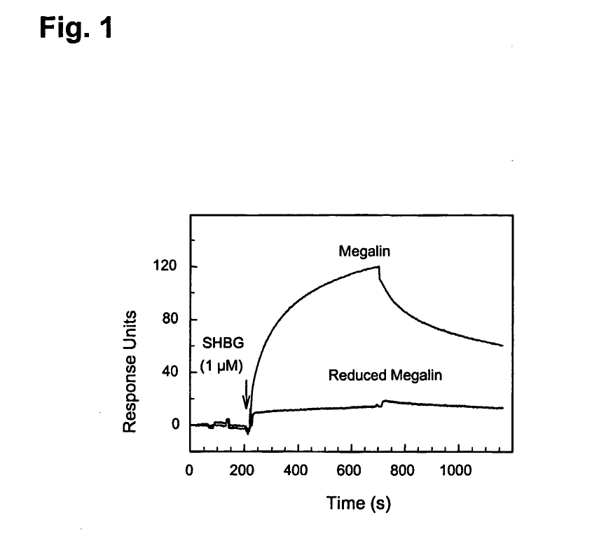

Purified native megalin (51.4 fmol/mm²) and reduced megalin (43.8 fmol/mm²) were immobilised on sensor chips, and the on and off rates for binding of 1 μM testosterone/SHBG complexes were recorded. The results are shown in FIG. 1. Nonspecific response from a blank chip exposed to a similar flow with the ligand have been subtracted. As shown, the testosterone/SHBG complex bound in a reversible manner to native megalin. The calculated affinity is approximately 140 nM. By comparison, no detectable binding was observed to the reduced and denatured receptor. In conclusion, complexes between testosterone and SHBG can bind megalin with high affinity.

### Example 2

Receptor-mediated uptake of dihydrotestosterone/SHBG complexes into BN 16 cells and keratinocytes.

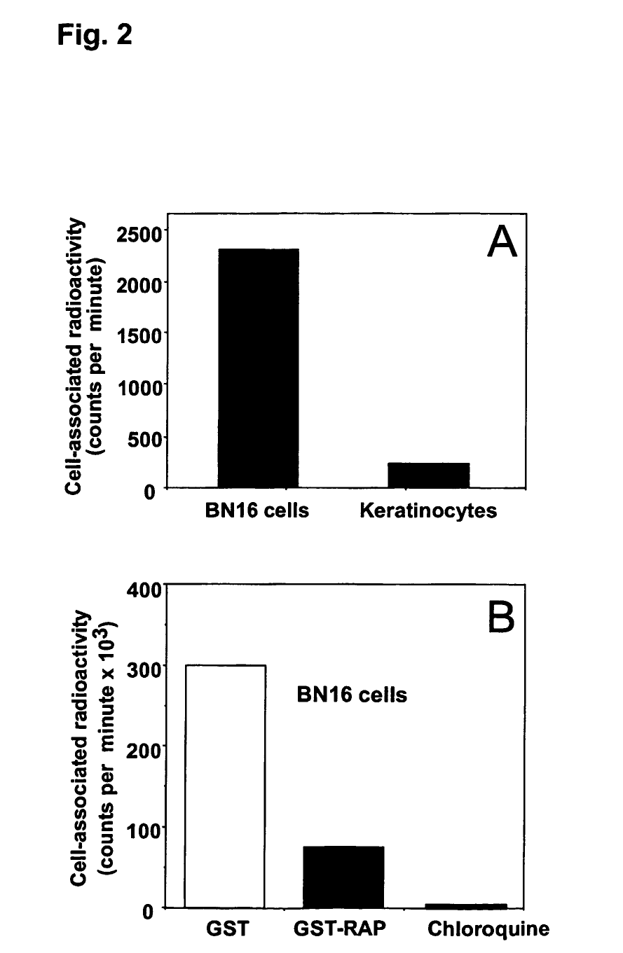

Brown Norway yolk sac carcinoma cells (BN16 cells) expressing megalin and primary cultures of human keratinocytes devoid of megalin were incubated with complexes of [³H]-dihydrotestosterone (DHT) and sex hormone binding globulin (SHBG). After 1 hr of incubation at 37° C., the cells were washed and the amount of cell-associated [³H]-DHT determined. As shown in FIG. 2A only the megalin expressing BN16 cells efficiently take up the steroid, whereas keratinocytes hardly take up any steroid.

BN16 cells were incubated with complexes of [³H]-DHT and SHBG. In addition, the medium included gluthathion S-transferase (GST), a fusion protein of GST and the receptor-associated protein (GST-RAP) or chloroquine, an inhibitor of endocytosis. After 4 hours incubation at 37° C., the cells were washed and the amount of cell-associated [³H]-DHT determined. In the presence of GST, which does not interfere with receptor-mediated endocytosis, significant amounts of [³H]-DHT (300×10³ cpm) were detected in BN16 cells, indicating cellular uptake. In contrast, a 75% reduction in [³H]-DHT uptake (70×10³ cpm) was observed in the presence of the megalin inhibitor RAP. Similar effects were observed in the presence of chloroquine. In conclusion, these findings indicate megalin-mediated uptake of DHT/SHBG complexes into cells.

### Example 3

Histological analysis of wild type and megalin-deficient mouse.

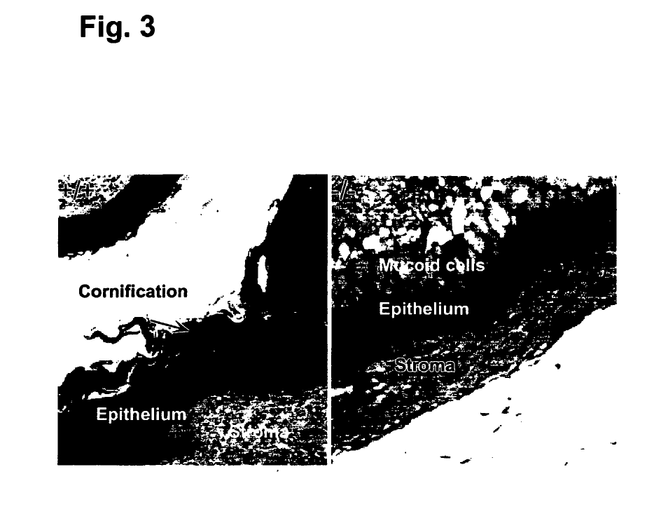

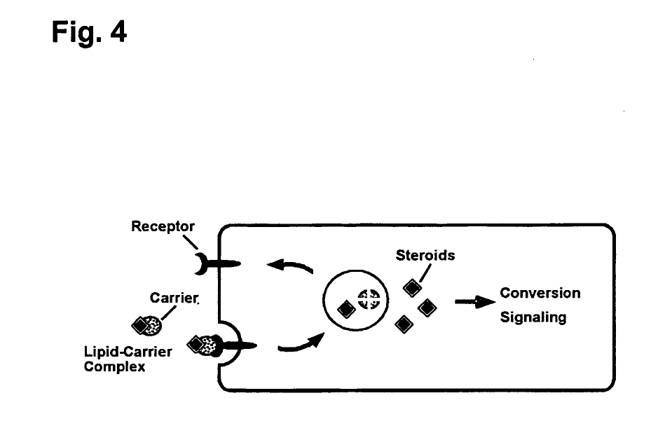

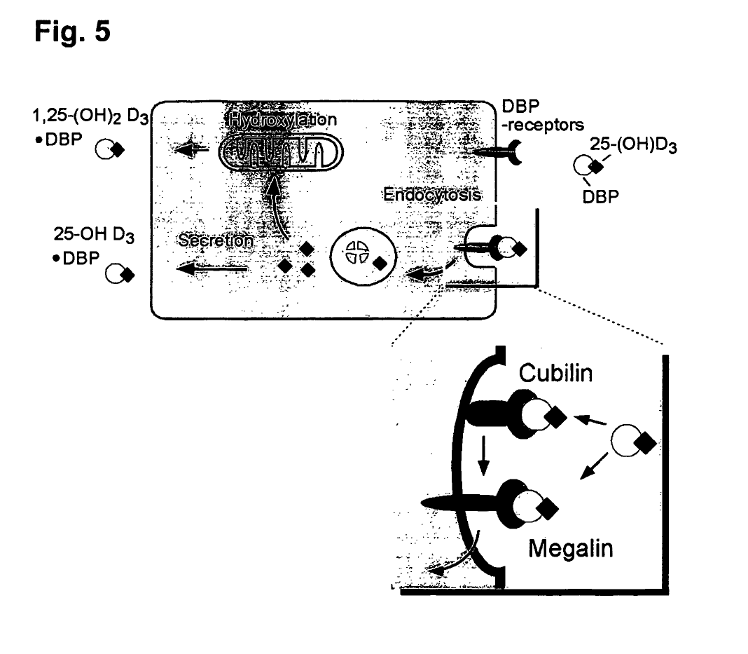

Vagina tissues were dissected from adult wild type (+/+) and megalin-deficient female mice (−/−) and prepared for routine paraffin embedding and sectioning. The tissue sections were stained with hematoxilin and eosin and are shown in FIG. 3. The wild type vagina illustrates normally developed stroma and hypertrophied epithelium. Note the estrogen-induced cornification of the epithelial surface that occurs during sexual maturation. In contrast, vagina tissue from receptor-deficient mice is characterized by a hypoplastic epithelium and lack of cornification. Instead, an additional layer of mucoid cells is detectable on the luminal surface of the epithelial cells. These alteration are consistent with a lack of estrogen-induced actions during puberty. In conclusion, the findings in the megalin knockout mice suggest a status of estrogen-insensitivity due to a lack of megalin-mediated uptake of estrogens.

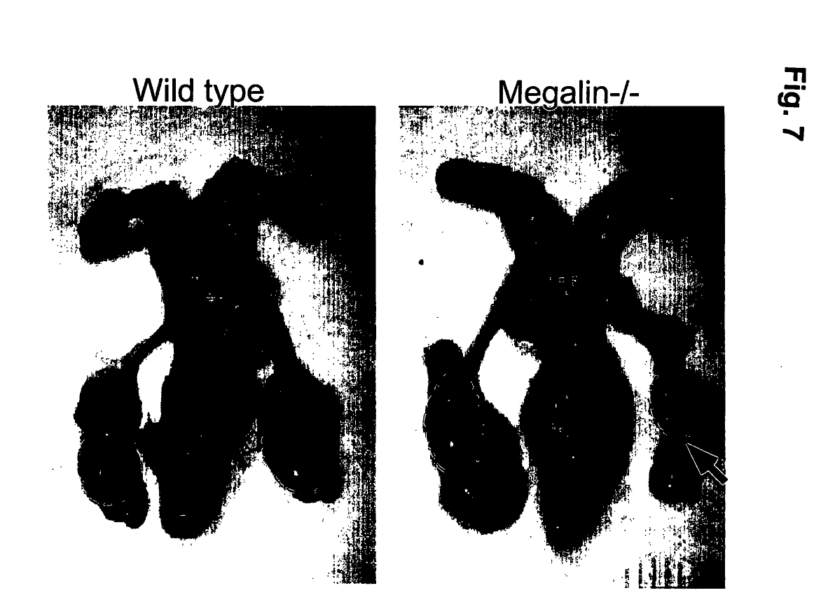

The urogenital tract was dissected from adult wild type or megalin-deficient male mice (megalin^{-/-}). The urogenital tracts are depicted in FIG. 7. The left testis of the receptor-deficient mice does not descent into the scrotum but remains in the abdominal cavity (unilateral cryptorchidism). Due to its exposure to a higher temperature in the body cavity, the non-descendent testis (arrow) regresses (as seen by its smaller size). Identical defects can be observed in rats treated with the nuclear androgen receptor antagonist flutamide (Zakaria et al., 2000). This finding suggests that megalin is responsible for uptake of androgens into the developing urogenital system and that absence of the receptor results in the lack of androgen signaling.

### Example 4

Competition of SHBG for Membrane-Binding

To test whether recombinant SHBG (rSHBG) interacts with SHBG receptors and in particular whether rSHBG can act as an inhibitor of SHBG binding to an SHBG receptor, the binding of rSHBG to bovine epididymis and endometrium membranes permeabilized in digitonin was assayed. Binding was performed in the presence of testosterone, to achieve steroid hormone-steroid hormone binding protein complex formation.

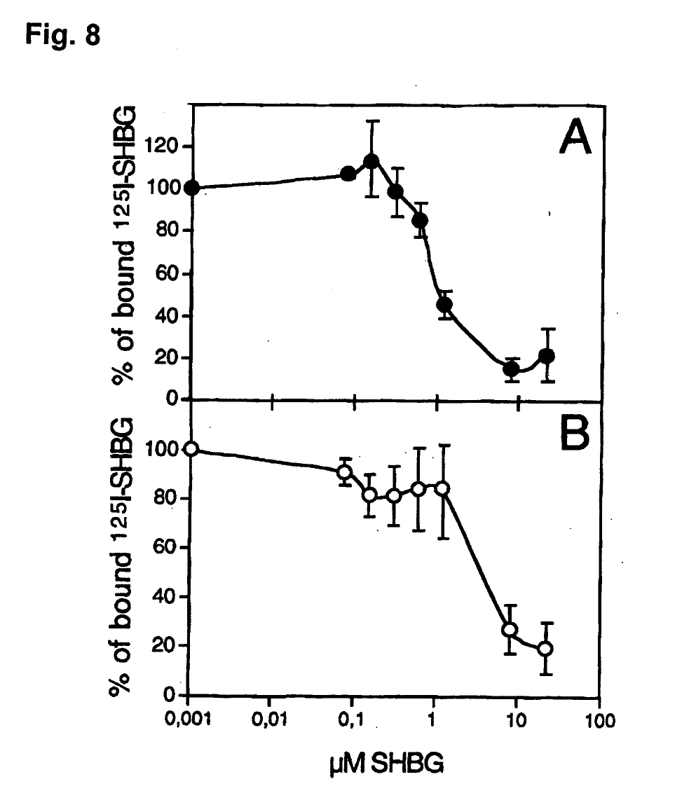

Briefly, purified recombinant SHBG was labelled with ¹²⁵I using the Iodo-Gen™ method [specific radioactivity (13-15)×10³ c.p.m./ng]. 400 pg of ¹²⁵I—SHBG was mixed with 600 μg of purified membranes (either bovine epididymis or bovine endometrium membranes permeabilized in digitonin) in a total volume of 200 μl of reaction buffer [10 mM Hepes, pH 7.4, 2 mM CaCl₂, 1 mM MgCl₂, 1% (w/v) BSA, 1 lM ZnCl₂, 11M testosterone and 301 g/ml digitonin]. In addition, the reaction mixture included the concentrations of unlabelled SHBG indicated in FIG. 8. The reaction mixture was incubated at 4° C. for 18 h and then precipitated on Millipore filters (GVWP 2932A) using a vacuum suction device. The filters were washed extensively with reaction buffer and the amount of bound radioactivity was determined.

As seen in FIG. 8, saturable binding of ¹²⁵I-labelled rSHBG to both membrane preparations was observed. Values are given as a percentage of ²⁵¹I-rSHBG bound in the absence of competitor (100%). The data represent the means of four individual experiments (±S.E.M.). Where no error bar is shown, the values are smaller than the actual symbol. The affinity of binding to the permeabilized epididymis membranes was in perfect agreement with published data for the native protein (Kd 0.5 μM).

### Example 5

Detection of Megalin in Prostate

Normal prostate tissue was dissected from dog and from rat males. The tissues were prepared for routine paraffin embedding and sectioning. The tissue sections were stained with goat anti-megalin antibodies followed by peroxidase-conjugated antigoat IgG. Bound IgG was visualized with diaminobenzidine.

FIG. 9 depicts the immunohistological detection of megalin in dog and rat prostate. The arrows denote localization of the receptor on the surface of epithelial cells. Expression of the receptor in these cell type suggests that it may be involved in benign hyperplasia and malignant prostate cancer. In particular, expression of megalin in dog prostate is interesting, because this dog model develops prostate hyperplasia and prostate cancer.

### Example 6

Tissue Specific Knock-Out of Megalin

Below is given an example of a model for testing the role of a steroid hormone binding protein receptor/co-receptor in a specific tissue. The model may be employed with any steroid hormone binding protein receptor/co-receptor, however the example describes tissue specific knock-out of megalin.

In order to further substantiate the role of megalin in steroid hormone metabolism, mice lacking the receptor in specific steroid-responsive tissues (e.g., uterus, epididymus, prostate, breast) using conditional or tissue-specific gene targeting is generated. Lack of the receptor in such tissues should result in a phenotype identical to one seen in mice with genetic or pharmacological defects of androgen or estrogen receptors.

To produce mice with a megalin gene defect in steroid-responsive tissues, a mouse line is generated carrying a megalin gene tagged with two adjacent recombination sites (IoxP sites). This mouse line (megalin^{flox/flox}) is crossed with mice carrying a Cre-recombinase transgene under breast-, prostate- or epididymus-specific promoter elements (Cre⁺) to produce mice doubly transgenic for megalin^{flox/flox} and Cre⁺(megalin^{flox/flox}/Cre⁺). Cre recombinase is a bacterial enzyme that recombines two adjacent IoxP sites to delete the intermittent sequence elements. Thus, tissue specific expression of Cre recombinase in breast, epididymis or prostate of megalin^{flox/flox}/Cre⁺ mice results in organ-specific deletion of the receptor gene.

### REFERENCES

Birn, H. Verroust, P. J., Nexo, E., Hager, H., Jacobsen, C., Christensen, E. I. & Moestrup, S. K. (1997) J. Biol. Chem. 272, 26497-26504.

Feldman, M., Lea, 0. A., Petrusz, P., Tres, L. L., Kierszenbaum, A. L. and French, F. S. (1981) Androgen-binding protein. Purfication from rat epididymis, characterization, and immunocytochemical localization. J. Biol. Chem. 256, 5170-5175.

Frairia, R., Fortunati, N., Berta, L., Fazzari, A., Fissore, F. and Gaidano, G. (1991) Sex steroid binding protein (SBP) receptors in estrogen sensitive tissues. J. Steroid Biochem. Mol. Biol. 40, 805-812

French, F. S. and Ritzen, E. M. (1973) A high-affinity androgen-binding protein (ABP) in rat testis: evidence for secretion into efferent duct fluid and absorption by epididymis. Endocrinology 93, 88-95.

Gerard, A., Khanfri, J., Gueant, J. L., Fremont, S., Nicolas, J. P., Grignon, G. and Gerard, H. (1988) Electron microscope radioautographic evidence of in vivo androgen-binding protein internalization in the rat epididymis principal cells. Endocrinology 122, 1297-1307.

Moestrup, S. K., Kozyraki, R., Kristiansen, M., Kaysen, J. H., Rasmussen, H. H., Brault, D., Pontillon, F., Goda, F. O., Christensen, E. I., Hammond, T. G., et al. (1998) J. Biol. Chem. 273, 5235-5242.

Anders Nykjaer* †, John C. Fyfe ‡, Renata Kozyraki*, Jo\ddot{} rg-Robert Leheste §, Christian Jacobsen*, Morten S. Nielsen*, Pierre J. Verroust ¶, Maria Aminoff_Albert de la Chapelle**, Søoren K. Moestrup*, Rahul Ray ††, Jørgen Gliemann*, Thomas E. Willnow §, and Erik I. Christensen ‡‡, 2001, Cubilin dysfunction causes abnormal metabolism of the steroid hormone 25(OH) vitamin D3; PNAS, vol. 98, no.24, p. 13895-13900

Zakaria et al., Comparative studies of fertility and histological development of contralateral scrotal testes in two rat models of unilateral cryptorchidism. (2000) Pediatr. Surg. Int. 16: 498-501.

1. An in vitro method for screening for a compound which alters uptake of steroid hormones into cells presenting a steroid hormone binding protein receptor, said method comprising
a) providing an assay for measuring binding to or uptake by the steroid hormone binding protein receptor of steroid hormones bound to or complexed with a steroid hormone binding protein,
b) adding the compound to be tested to the assay, and
c) determining the amount of steroid hormones bound to or complexed with a steroid hormone binding protein which is bound to or taken up by the steroid hormone binding protein receptor,
d) comparing the amount determined in step c) with an amount measured in the absence of the compound to be tested,
e) wherein a difference in the two amounts identifies a compound which alters the binding or uptake of steroid hormones bound to or complexed with steroid hormone binding proteins.

2. The method according to claim 1, wherein the steroid hormone is selected from the group consisting of androgens, estrogens, progestogens and corticoids.

3. The method according to claim 1, wherein the steroid hormone is selected from the group consisting of androgens and estrogens.

4. The method according to claim 1, wherein the steroid hormone is selected from the group consisting of testosterone, dihydrotestosterone, estrion and estradiol.

5. The method according to claim 1, wherein the steroid hormone binding protein is selected from the group consisting of sex hormone binding globulin, corticosteroid-binding globulin, Clara cell secretory protein (CCSP), apolipoprotein D (apoD) and 24p3.

6. The method according to claim 1, wherein the steroid hormone binding protein is the sex hormone binding globulin.

7. The method according to claim 1, wherein the steroid hormone binding protein is a human steroid hormone binding protein.

8. The method according to claim 1, wherein the steroid hormone binding protein receptor is selected from a group of receptors of low density lipoprotein (LDL) receptor gene family, said group comprising VLDL receptor, LRP-1B, MEGF-7, ApoE receptor-2, chicken VTG receptor, Drosophila Y1 protein, LDL receptor-related protein, gp 330/megalin and C. elegans gp330/megalin-related protein receptor.

9. The method according to claim 1, wherein the steroid hormone binding protein receptor is selected from the group consisting of LRP, megalin, VLDL receptor, MGEF-7, LRP1B and ApoE receptor-2.

10. The method according to claim 1, wherein the steroid hormone binding protein receptor is selected from the group consisting of ApoE receptor-2 and megalin.

11. The method according to claim 1, wherein the steroid hormone binding protein receptor is megalin.

12. The method according to claim 1, wherein the steroid hormone binding protein receptor is a human steroid hormone binding receptor.

13. The method according to claim 1, wherein the cells presenting the steroid hormone binding protein receptors are selected from the group consisting of prostate cells, cells in epididymis, endometrial cells, ovarial cells, breast parenchymal cells, prostate carcinoma cells and breast carcinoma cells.

14. The method according to claim 1, wherein the assay is a non-radioactive microtiter plate assay using purified steroid hormone binding protein receptors or fragments thereof immobilized on the plate surface.

15. The method according to claim 1, wherein the assay comprises the steps of
a) providing a non-radioactive microtiter plate having purified steroid hormone binding protein receptors and/or steroid hormone binding protein co-receptors or fragments thereof immobilised on the plate surface,
b) incubating the plate with the compound to be tested, and
c) incubating the plate with fluorescent labelled steroid binding proteins or fragments thereof and/or with fluorescent labelled steroid hormones,
d) determining the amount of fluorescence of the plate.

16. The method according to claim 1, wherein the assay comprises the steps of
a) providing a cell which comprises a steroid hormone protein binding receptor, a steroid hormone nuclear receptor and a first nucleic acid sequence encoding a directly or indirectly detectable protein, said first nucleic acid sequence being operably linked to a second nucleic acid sequence, which can direct transcription depending on the presence of steroid hormone nuclear receptor complexed with steroid hormone.
b) detecting said detectable protein.
c) correlating the amount of detectable protein to the uptake of steroid hormone into said cell.

17. The method according to claim 1, wherein the compound to be tested is selected from steroid hormone binding protein receptor domains or fragments thereof, natural steroid hormone binding protein receptor ligands, modified steroid hormone binding proteins or fragments thereof, steroid hormone binding protein receptor antagonists, such as receptor associated protein, or small organic molecules.

18. The method according to claim 1, wherein the compound is selected from a library of naturally occurring and synthetic compounds which are randomly tested for alteration of the binding.

19. The method according to claim 17, wherein the steroid hormone binding protein receptor domain is capable of binding a steroid hormone binding protein.

20. The method according to claim 17, wherein the steroid hormone binding protein receptor domain is selected from steroid hormone binding protein receptor domains which comprise at least one complement type repeat.

21. The method according to claim 17, wherein the steroid hormone binding protein receptor domain is selected from the group consisting of fragments of the MGEF-7 polypeptide sequence (SEQ ID NO: 2), fragments of the megalin polypeptide sequence (SEQ ID NO: 3), fragments of the LRP1B polypeptide sequence (SEQ ID NO: 4), fragments of the ApoE receptor 2 polypeptide sequence (SEQ ID NO: 5), fragments of the LRP polypeptide sequence (SEQ ID NO: 6) and fragments of the VLDL receptor polypeptide sequence (SEQ ID NO: 7).

22. The method according to claim 1, wherein the assay includes a cell expressing the steroid hormone binding protein receptor and the compound is a nucleic acid sequence which potentially alters the expression of the receptor.

23. The method according to claim 1, wherein the compound competitively inhibits the binding or complexing of a steroid hormone to a steroid hormone binding protein.

24. The method according to claim 1, wherein the compound competitively inhibits the binding of a steroid hormone binding protein to a steroid hormone binding protein receptor.

25. The method according to claim 1, wherein the compound competitively inhibits the binding of a steroid hormone binding protein to a steroid hormone binding protein co-receptor.

26. The method according to claim 1, wherein the compound increases the uptake of the steroid hormone.

27. The method according to claim 26, wherein the compound alters dimerisation of steroid hormone binding proteins.

28. A compound which alters the uptake of steroid hormone into cells presenting a steroid hormone binding protein receptor as identified by the method in claim 1.

29. The compound of claim 28, selected from steroid hormone binding protein receptor domains or fragments thereof, natural steroid hormone binding protein receptor ligands, steroid hormone binding protein co-receptor domains and fragments thereof, modified steroid hormone binding proteins or fragments thereof, fragments of steroid hormone binding proteins, steroid hormone binding protein receptor antagonists, such as receptor associated protein (RAP), or small organic molecules.

30. The compound according to claim 28, wherein the compound is selected from a library of naturally occurring and synthetic compounds which are randomly tested for alteration of the binding.

31. The compound according to claim 29, wherein the steroid hormone binding protein receptor domain is capable of binding a steroid hormone binding protein.

32. The compound according to claim 29, wherein the steroid hormone binding protein receptor domain is selected from steroid hormone binding protein receptor domains which comprise at least one complement type repeat.

33. The compound according to claim 29, wherein the steroid hormone binding protein receptor domain is selected from the group consisting of fragments of the VLDL receptor polypeptide sequence (SEQ ID NO: 7), fragments of the megalin polypeptide sequence (SEQ ID NO: 3) and fragments of the ApoE receptor 2 polypeptide sequence (SEQ ID NO: 5).

34. The compound according to claim 29, wherein the steroid hormone binding protein co-receptor domain is selected from the group consisting of fragments of the cubilin polypeptide sequence (SEQ ID NO: 8).

35. The compound according to claim 28, wherein the compound competitively inhibits the binding or complexing of a steroid hormone to a steroid hormone binding protein.

36. The compound according to claim 28, wherein the compound competitively inhibits the binding of a steroid hormone binding protein to a steroid hormone binding protein receptor.

37. The compound according to claim 29, wherein said fragment is capable of associating with a steroid hormone binding protein receptor and furthermore said fragment is not capable of associating with a steroid hormone.

38. The compound according to claim 28, wherein said compound is a fragment of a steroid hormone binding protein capable of associating with a steroid hormone binding protein receptor, wherein said fragment is selected from the group consisting of:
i. fragments consisting of amino acid 47 to 167 of human SHBG (SEQ ID NO: 1); and
ii. fragments consisting of amino acid 128 to 137 of human SHBG (SEQ ID NO: 1); and
iii. fragments consisting of amino acid 106 to 125 of human SHBG (SEQ ID NO:1); and
iv. functional homologues of fragments of i) to iii), consisting of fragments at least 90% homologues to the fragments of i) to iii); and
v. functional homologues of fragments of i) to iii), consisting of said fragments or fragments at least 90% homologues thereto with addition of in the range of 2 to 50 amino acids.

39. The compound according to claim 39, wherein said functional homologues consists of fragments at least 90% homologues to the fragments of i), wherein amino acid residues 47, 106, 123, 125, 166 and 167 of SEQ ID No: 1 are selected from the group consisting of Arg and Lys.

40. The compound according to claim 39, wherein said functional homologues consists of fragments at least 90% homologues to the fragments of iii), wherein amino acid residues 106, 123, 125 of SEQ ID No: 1 are selected from the group consisting of Arg and Lys.

41. The compound according to claim 28 wherein said compound is selected from fragments of RAP that can associate with a steroid hormone binding protein receptor.

42. The compound according to claim 28, wherein said compound is a nucleic acid sequence selected from the group consisting of a DNA sequence encoding for an antisense RNA of a steroid hormone binding protein receptor and an antisense RNA of a steroid hormone binding protein receptor.

43. The compound according to claim 28, wherein the compound competitively inhibits the binding of a steroid hormone binding protein to a steroid hormone binding protein co-receptor.

44. The compound according to claim 28, wherein the compound alters the uptake of the steroid hormone.

45. The compound according to claim 28, wherein the compound alters dimerisation of steroid hormone binding proteins.

46. A method for determining the effect of a compound on uptake of steroid hormones into cells presenting a steroid hormone binding protein receptor, said method comprising the steps of
a) administering said compound to a mammal naturally expressing the receptor,
b) measuring expression of receptor mRNA or protein in tissues of the mammal or measuring receptor-mediated binding or transport of steroid hormones bound to or complexed with steroid hormone binding proteins or measuring uptake of steroid hormones or steroid hormone/steroid hormone binding protein complexes into cells of said mammal,
c) comparing the measurement of step b) with a measurement measured in the absence of the compound to be tested,
d) wherein the difference in the two measurements identifies the effect of said compound on the uptake of steroid hormones into cells presenting a steroid hormone binding protein receptor.

47. The method of claim 47 wherein said compound is selected from steroid hormone binding protein receptor domains or fragments thereof, natural steroid hormone binding protein receptor ligands, steroid hormone binding protein co-receptor domains and fragments thereof, modified steroid hormone binding proteins or fragments thereof, fragments of steroid hormone binding proteins, steroid hormone binding protein receptor antagonists, such as receptor associated protein (RAP), or small organic molecules.

48. The method according to claim 46, wherein step b) comprise an assay involving the use of immunoreactive species.

49. The method according to claim 46, wherein step b) comprise an assay involving the use of one or more purified species selected from the group consisting of steroid hormones, steroid hormone binding proteins, steroid hormone binding protein receptors, steroid hormone binding protein co-receptors and fragments thereof and functional homologues thereof.

50. The method according to claim 46, wherein said method further comprises administering said compound to a mammal lacking expression of said steroid hormone binding protein receptor.

51. The method according to claim 50, wherein said mammal only lacks expression of said steroid hormone binding protein receptor in one or more selected tissues.

52. A method of treating a clinical condition comprising administering to an individual in need thereof a sufficient amount of a compound comprising a steroid hormone binding protein receptor domain is selected from the group consisting of fragments of the VLDL receptor polypeptide sequence (SEQ ID NO: 7), fragments of the megalin polypeptide sequence (SEQ ID NO: 3), fragments of the ApoE receptor 2 polypeptide sequence (SEQ ID NO: 5), and combinations thereof.

53. A method of treating prostate cancer comprising administering to an individual having prostate cancer a sufficient amount of a compound selected from steroid hormone binding protein receptor domains or fragments thereof, natural steroid hormone binding protein receptor ligands, steroid hormone binding protein co-receptor domains and fragments thereof, modified steroid hormone binding proteins or fragments thereof, fragments of steroid hormone binding proteins, steroid hormone binding protein receptor antagonists, such as receptor associated protein (RAP), or small organic molecules.

54. A method of treating breast cancer comprising administering to an individual having breast cancer a sufficient amount of a compound comprising a steroid hormone binding protein receptor domain is selected from the group consisting of fragments of the VLDL receptor polypeptide sequence (SEQ ID NO: 7), fragments of the megalin polypeptide sequence (SEQ ID NO: 3), fragments of the ApoE receptor 2 polypeptide sequence (SEQ ID NO: 5), and combinations thereof.

55. A method of preventing pregnancy comprising administering to an individual in need thereof a sufficient amount of a compound selected from steroid hormone binding protein receptor domains or fragments thereof, natural steroid hormone binding protein receptor ligands, steroid hormone binding protein co-receptor domains and fragments thereof, modified steroid hormone binding proteins or fragments thereof, fragments of steroid hormone binding proteins, steroid hormone binding protein receptor antagonists, such as receptor associated protein (RAP), or small organic molecules.

56. The method according to claim 55, wherein said individual is a male.

57. A pharmaceutical composition comprising a compound comprising a steroid hormone binding protein receptor domain is selected from the group consisting of fragments of the VLDL receptor polypeptide sequence (SEQ ID NO: 7), fragments of the megalin polypeptide sequence (SEQ ID NO: 3), fragments of the ApoE receptor 2 polypeptide sequence (SEQ ID NO: 5), and combinations thereof.

58-60 (Cancelled)


SEQUENCE LISTING (8 sequences)

SEQ ID NO: 1 - protein, 402 aa, Homo sapiens
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQKVVLSSGSGPGLDLPLVLGLPLQLKLSMSRVVLSQGSKMKALALPPLGLAPLLNLWAKPQGRLFLGALPGEDSSTSFCLNGLWAQGQRLDVDQALNRSHEIWTHSCPQSPGNGTDASH

SEQ ID NO: 2 - protein, 1576 aa, Homo sapiens
LCNGVNDCGDNSDESPQQNCRPRTGEENCNVNNGGCAQKCQMVRGAVQCTCHTGYRLTEDGHTCQDVNECAEEGYCSQGCTNSEGAFQCWCETGYELRPDRRSCKALGPEPVLLFANRIDIRQVLPHRSEYTLLLNNLENAIALDFHHRRELVFWSDVTLDRILRANLNGSNVEEVVSTGLESPGGLAVDWVHDKLYWTDSGTSRIEVANLDGAHRKVLLWQNLEKPRAIALHPMEGTIYWTDWGNTPRIEASSMDGSGRRIIADTHLFWPNGLTIDYAGRRMYWVDAKHHVIERANLDGSHRKAVISQGLPHPFAITVFEDSLYWTDWHTKSINSANKFTGKNQEIIRNKLHFPMDIHTLHPQRQPAGKNRCGDNNGGCTHLCLPSGQNYTCACPTGFRKISSHACAQSLDKFLLFARRMDIRRISFDTEDLSDDVIPLADVRSAVALDWDSRDDHVYWTDVSTDTISRAKWDGTGQEVVVDTSLESPAGLAIDWVTNKLYWTDAGTDRIEVANTDGSMRTVLIWENLDRPRDIVVEPMGGYMYWTDWGASPKIERAGMDASGRQVIISSNLTWPNGLAIDYGSQRLYWADAGMKTIEFAGLDGSKRKVLIGSQLPHPFGLTLYGERIYWTDWQTKSIQSADRLTGLDRETLQENLENLMDIHVFHRRRPPVSTPCAMENGGCSHLCLRSPNPSGFSCTCPTGINLLSDGKTCSPGMNSFLIFARRIDIRMVSLDIPYFADVVVPINITMKNTIAVGVDPQEGKVYWSDSTLHRISRANLDGSQHEDIITTGLQTTDGLAVDAIGRKVYWTDTGTNRIEVGNLDGSMRKVLVWQNLDSPRAIVLYHEMGFMYWTDWGENAKLERSGMDGSDRAVLINNNLGWPNGLTVDKASSQLLWADAHTERIEAADLNGANRHTLVSPVQHPYGLTLLDSYIYWTDWQTRSIHRADKGTGSNVILVRSNLPGLMDMQAVDRAQPLGFNKCGSRNGGCSHLCLPRPSGFSCACPTGIQLKGDGKTCDPSPETYLLFSSRGSIRRISLDTSDHTDVHVPVPELNNVISLDYDSVDGKVYYTDVFLDVIRRADLNGSNMETVIGRGLKTTDGLAVDWVARNLYWTDTGRNTIEASRLDGSCRKVLINNSLDEPRAIAVFPRKGYLFWTDWGHIAKIERANLDGSERKVLINTDLGWPNGLTLDYDTRRIYWVDAHLDRIESADLNGKLRQVLVGHVSHPFALTQQDRWIYWTDWQTKSIQRVDKYSGRNKETVLANVEGLMDIIVVSPQRQTGTNACGVNNGGCTHLCFARASDFVCACPDEPDSQPCSLVPGLVPPAPRATGMSEKSPVLPNTPPTTLYSSTTRTRTSLEEVEGRCSERDARLGLCARSNDAVPAAPGEGLHISYAIGGLLSILLILVVIAALMLYRHKKSKFTDPGMGNLTYSNPSYRTSTQEVKIEAIPKPAMYNQLCYKKEGGPDHNYTKEKIKIVEGICLLSGDDAEWDDLKQLRSSRGGLLRDHVCMKTDTVSIQASSGSLDDTEMEQLLQEEQSECSSVHTAATPERRGSLPDTGWKHERKLSSESQV

SEQ ID NO: 3 - protein, 4655 aa, Homo sapiens
MDRGPAAVACTLLLALVACLAPASGQECDSAHFRCGSGHCIPADWRCDGTKDCSDDADEIGCAVVTCQQGYFKCQSEGQCIPSSWVCDQDQDCDDGSDERQDCSQSTCSSHQITCSNGQCIPSEYRCDHVRDCPDGADENDCQYPTCEQLTCDNGACYNTSQKCDWKVDCRDSSDEINCTEICLHNEFSCGNGECIPRAYVCDHDNDCQDGSDEHACNYPTCGGYQFTCPSGRCIYQNWVCDGEDDCKDNGDEDGCESGPHDVHKCSPREWSCPESGRCISIYKVCDGILDCPGREDENNTSTGKYCSMTLCSALNCQYQCHETPYGGACFCPPGYIINHNDSRTCVEFDDCQIWGICDQKCESRPGRHLCHCEEGYILERGQYCKANDSFGEASIIFSNGRDLLIGDIHGRSFRILVESQNRGVAVGVAFHYHLQRVFWTDTVQNKVFSVDINGLNIQEVLNVSVETPENLAVDWVNNKIYLVETKVNRIDMVNLDGSYRVTLITENLGHPRGIAVDPTVGYLFFSDWESLSGEPKLERAFMDGSNRKDLVKTKLGWPAGVTLDMISKRVYWVDSRFDYIETVTYDGIQRKTVVHGGSLIPHPFGVSLFEGQVFFTDWTKMAVLKANKFTETNPQVYYQASLRPYGVTVYHSLRQPYATNPCKDNNGGCEQVCVLSHRTDNDGLGFRCKCTFGFQLDTDERHCIAVQNFLIFSSQVAIRGIPFTLSTQEDVMVPVSGNPSFFVGIDFDAQDSTIFFSDMSKHMIFKQKIDGTGREILAANRVENVESLAFDWISKNLYWTDSHYKSISVMRLADKTRRTVVQYLNNPRSVVVHPFAGYLFFTDWFRPAKIMRAWSDGSHLLPVINTTLGWPNGLAIDWAASRLYWVDAYFDKIEHSTFDGLDRRRLGHIEQMTHPFGLAIFGEHLFFTDWRLGAIIRVRKADGGEMTVIRSGIAYILHLKSYDVNIQTGSNACNQPTHPNGDCSHFCFPVPNFQRVCGCPYGMRLASNHLTCEGDPTNEPPTEQCGLFSFPCKNGRCVPNYYLCDGVDDCHDNSDEQLCGTLNNTCSSSAFTCGHGECIPAHWRCDKRNDCVDGSDEHNCPTHAPASCLDTQYTCDNHQCISKNWVCDTDNDCGDGSDEKNCNSTETCQPSQFNCPNHRCIDLSFVCDGDKDCVDGSDEVGCVLNCTASQFKCASGDKCIGVTNRCDGVFDCSDNSDEAGCPTRPPGMCHSDEFQCQEDGICIPNFWECDGHPDCLYGSDEHNACVPKTCPSSYFHCDNGNCIHRAWLCDRDNDCGDMSDEKDCPTQPFRCPSWQWQCLGHNICVNLSVVCDGIFDCPNGTDESPLCNGNSCSDFNGGCTHECVQEPFGAKCLCPLGFLLANDSKTCEDIDECDILGSCSQHCYNMRGSFRCSCDTGYMLESDGRTCKVTASESLLLLVASQNKIIADSVTSQVHNIYSLVENGSYIVAVDFDSISGRIFWSDATQGKTWSAFQNGTDRRVVFDSSIILTETIAIDWVGRNLYWTDYALETIEVSKIDGSHRTVLISKNLTNPRGLALDPRMNEHLLFWSDWGHHPRIERASMDGSMRTVIVQDKIFWPCGLTIDYPNRLLYFMDSYLDYMDFCDYNGHHRRQVIASDLIIRHPYALTLFEDSVYWTDRATRRVMRANKWHGGNQSVVMYNIQWPLGIVAVHPSKQPNSVNPCAFSRCSHLCLLSSQGPHFYSCVCPSGWSLSPDLLNCLRDDQPFLITVRQHIIFGISLNPEVKSNDAMVPIAGIQNGLDVEFDDAEQYIYWVENPGEIHRVKTDGTNRTVFASISMVGPSMNLALDWISRNLYSTNPRTQSIEVLTLHGDIRYRKTLIANDGTALGVGFPIGITVDPARGKLYWSDQGTDSGVPAKIASANMDGTSVKTLFTGNLEHLECVTLDIEEQKLYWAVTGRGVIERGNVDGTDRMILVHQLSHPWGIAVHDSFLYYTDEQYEVIERVDKATGANKIVLRDNVPNLRGLQVYHRRNAAESSNGCSNNMNACQQICLPVPGGLFSCACATGFKLNPDNRSCSPYNSFIVVSMLSAIRGFSLELSDHSETMVPVAGQGRNALHVDVDVSSGFIYWCDFSSSVASDNAIRRIKPDGSSLMNIVTHGIGENGVRGIAVDWVAGNLYFTNAFVSETLIEVLRINTTYRRVLLKVTVDMPRHIVVDPKNRYLFWADYGQRPKIERSFLDCTNRTVLVSEGIVTPRGLAVDRSDGYVYWVDDSLDIIARIRINGENSEVIRYGSRYPTPYGITVFENSIIWVDRNLKKIFQASKEPENTEPPTVIRDNINWLRDVTIFDKQVQPRSPAEVNNNPCLENNGGCSHLCFALPGLHTPKCDCAFGTLQSDGKNCAISTENFLIFALSNSLRSLHLDPENHSPPFQTINVERTVMSLDYDSVSDRIYFTQNLASGVGQISYATLSSGIHTPTVIASGIGTADGIAFDWITRRIYYSDYLNQMINSMAEDGSNRTVIARVPKPRAIVLDPCQGYLYWADWDTHAKIERATLGGNFRVPIVNSSLVMPSGLTLDYEEDLLYWVDASLQRIERSTLTGVDREVIVNAAVHAFGLTLYGQYIYWTDLYTQRIYRANKYDGSGQIAMTTNLLSQPRGINTVVKNQKQQCNNPCEQFNGGCSHICAPGPNGAECQCPHEGNWYLANNRKHCIVDNGERCGASSFTCSNGRCISEEWKCDNDNDCGDGSDEMESVCALHTCSPTAFTCANGRCVQYSYRCDYYNDCGDGSDEAGCLFRDCNATTEFMCNNRRCIPREFICNGVDNCHDNNTSDEKNCPDRTCQSGYTKCHNSNICIPRVYLCDGDNDCGDNSDENPTYCTTHTCSSSEFQCASGRCIPQHWYCDQETDCFDASDEPASCGHSERTCLADEFKCDGGRCIPSEWICDGDNDCGDMSDEDKRHQCQNQNCSDSEFLCVNDRPPDRRCIPQSWVCDGDVDCTDGYDENQNCTRRTCSENEFTCGYGLCIPKIFRCDRHNDCGDYSDERGCLYQTCQQNQFTCQNGRCISKTFVCDEDNDCGDGSDELMHLCHTPEPTCPPHEFKCDNGRCIEMMKLCNHLDDCLDNSDEKGCGINECHDPSISGCDHNCTDTLTSFYCSCRPGYKLMSDKRTCVDIDECTEMPFVCSQKCENVIGSYICKCAPGYLREPDGKTCRQNSNIEPYLIFSNRYYLRNLTIDGYFYSLILEGLDNVVALDFDRVEKRLYWIDTQRQVIERMFLNKTNKETIINHRLPAAESLAVDWVSRKLYWLDARLDGLFVSDLNGGHRRMLAQHCVDANNTFCFDNPRGLALHPQYGYLYWADWGHRAYIGRVGMDGTNKSVIISTKLEWPNGITIDYTNDLLYWADAHLGYIEYSDLEGHHRHTVYDGALPHPFAITIFEDTIYWTDWNTRTVEKGNKYDGSNRQTLVNTTHRPFDIHVYHPYRQPIVSNPCGTNNGGCSHLCLIKPGGKGFTCECPDDFRTLQLSGSTYCMPMCSSTQFLCANNEKCIPIWWKCDGQKDCSDGSDELALCPQRFCRLGQFQCSDGNCTSPQTLCNAHQNCPDGSDEDRLLCENHHCDSNEWQCANKRCIPESWQCDTFNDCEDNSDEDSSHCASRTCRPGQFRCANGRCIPQAWKCDVDNDCGDHSDEPIEECMSSAHLCDNFTEFSCKTNYRCIPKWAVCNGVDDCRDNSDEQGCEERTCHPVGDFRCKNHHCIPLRWQCDGQNDCGDNSDEENCAPRECTESEFRCVNQQCIPSRWICDHYNDCGDNSDERDCEMRTCHPEYFQCTSGHCVHSELKCDGSADCLDASDEADCPTRFPDGAYCQATMFECKNHVCIPPYWKCDGDDDCGDGSDEELHLCLDVPCNSPNRFRCDNNRCIYSHEVCNGVDDCGDGTDETEEHCRKPTPKPCTEYEYKCGNGHCIPHDNVCDDADDCGDWSDELGCNKGKERTCAENICEQNCTQLNEGGFICSCTAGFETNVFDRTSCLDINECEQFGTCPQHCRNTKGSYECVCADGFTSMSDRPGKRCAAEGSSPLLLLPDNVRIRKYNLSSERFSEYLQDEEYIQAVDYDWDPKDIGLSVVYYTVRGEGSRFGAIKRAYIPNFESGRNNLVQEVDLKLKYVMQPDGIAVDWVGRHIYWSDVKNKRIEVAKLDGRYRKWLISTDLDQPAAIAVNPKLGLMFWTDWGKEPKIESAWMNGEDRNILVFEDLGWPTGLSIDYLNNDRIYWSDFKEDVIETIKYDGTDRRVIAKEAMNPYSLDIFEDQLYWISKEKGEVWKQNKFGQGKKEKTLVVNPWLTQVRIFHQLRYNKSVPNLCKQICSHLCLLRPGGYSCACPQGSSFIEGSTTECDAAIELPINLPPPCRCMHGGNCYFDETDLPKCKCPSGYTGKYCEMAFSKGISPGTTAVAVLLTILLIVVIGALAIAGFFHYRRTGSLLPALPKLPSLSSLVKPSENGNGVTFRSGADLNMDIGVSGFGPETAIDRSMAMSEDFVMEMGKQPIIFENPMYSARDSAVKVVQPIQVTVSENVDNKNYGSPINPSEIVPETNPTSPAADGTQVTKWNLFKRKSKQTTNFENPIYAQMENEQKESVAATPPPSPSLPAKPKPPSRRDPTPTYSATEDTFKDTANLVKEDSEV

SEQ ID NO: 4 - protein, 4599 aa, Homo sapiens
  misc_feature (4343)..(4343): Xaa can be any naturally occurring amino acid
MSEFLLALLTLSGLLPIARVLTVGADRDQQLCDPGEFLCHDHVTCVSQSWLCDGDPDCPDDSDESLDTCPEEVEIKCPLNHIACLGTNKCVHLSQLCNGVLDCPDGYDEGVHCQELLSNCQQLNCQYKCTMVRNSTRCYCEDGFEITEDGRSCKDQDECAVYGTCSQTCRNTHGSYTCSCVEGYLMQPDNRSCKAKIEPTDRPPILLIANFETIEVFYLNGSKMATLSSVNGNEIHTLDFIYNEDMICWIESRESSNQLKCIQITKAGGLTDEWTINILQSFHNVQQMAIDWLTRNLYFVDHVGDRIFVCNSNGSVCVTLIDLELHNPKAIAVDPIAGKLFFTDYGNVAKVERCDMDGMNRTRIIDSKTEQPAALALDLVNKLVYWVDLYLDYVGVVDYQGKNRHTVIQGRQVRHLYGITVFEDYLYATNSDNYNIVRINRFNGTDIHSLIKIENAWGIRIYQKRTQPTVRSHACEVDPYGMPGGCSHICLLSSSYKTRTCRCRTGFNLGSDGRSCKRPKNELFLFYGKGRPGIVRGMDLNTKIADEYMIPIENLVNPRALDFHAETNYIYFADTTSFLIGRQKIDGTERETILKDDLDNVEGIAVDWIGNNLYWTNDGHRKTINVARLEKASQSRKTLLEGEMSHPRGIVVDPVNGWMYWTDWEEDEIDDSVGRIEKAWMDGFNRQIFVTSKMLWPNGLTLDFHTNTLYWCDAYYDHIEKVFLNGTHRKIVYSGRELNHPFGLSHHGNYVFWTDYMNGSIFQLDLITSEVTLLRHERPPLFGLQIYDPRKQQGDNMCRVNNGGCSTLCLAIPGGRVCACADNQLLDENGTTCTFNPGEALPHICKAGEFRCKNRHCIQARWKCDGDDDCLDGSDEDSVNCFNHSCPDDQFKCQNNRCIPKRWLCDGANDCGSNEDESNQTCTARTCQVDQFSCGNGRCIPRAWLCDREDDCGDQTDEMASCEFPTCEPLTQFVCKSGRCISSKWHCDSDDDCGDGSDEVGCVHSCFDNQFRCSSGRCIPGHWACDGDNDCGDFSDEAQINCTKEEIHSPAGCNGNEFQCHPDGNCVPDLWRCDGEKDCEDGSDEKGCNGTIRLCDHKTKFSCWSTGRCINKAWVCDGDIDCEDQSDEDDCDSFLCGPPKHPCANDTSVCLQPEKLCNGKKDCPDGSDEGYLCDECSLNNGGCSNHCSVVPGRGIVCSCPEGLQLNKDNKTCEIVDYCSNHLKCSQVCEQHKHTVKCSCYEGWKLDVDGESCTSVDPFEAFIIFSIRHEIRRIDLHKRDYSLLVPGLRNTIALDFHFNQSLLYWTDVVEDRIYRGKLSESGGVSAIEVVVEHGLATPEGLTVDWIAGNIYWIDSNLDQIEVAKLDGSLRTTLIAGAMEHPRAIALDPRYGILFWTDWDANFPRIESASMSGAGRKTIYKDMKTGAWPNGLTVDHFEKRIVWTDARSDAIYSALYDGTNMIEIIRGHEYLSHPFAVSLYGSEVYWTDWRTNTLSKANKWTGQNVSVIQKTSAQPFDLQIYHPSRQPQAPNPCAANDGKGPCSHMCLINHNRSAACACPHLMKLSSDKKTCYEMKKFLLYARRSEIRGVDIDNPYFNFITAFTVPDIDDVTVIDFDASEERLYWTDIKTQTIKRAFINGTGLETVISRDIQSIRGLAVDWVSRNLYWISSEFDETQINVARLDGSLKTSIIHGIDKPQCLAAHPVRGKLYWTDGNTINMANMDGSNSKILFQNQKEPVGLSIDYVENKLYWISSGNGTINRCNLDGGNLEVIESMKEELTKATALTIMDKKLWWADQNLAQLGTCSKRDGRNPTILRNKTSGVVHMKVYDKEAQQGSNSCQLNNGGCSQLCLPTSETTRTCMCTVGYYLQKNRMSCQGIESFLMYSVHEGIRGIPLEPSDKMDALMPISGTSFAVGIDFHAENDTIYWTDMGFNKISRAKRDQTWKEDIITNGLGRVEGIAVDWIAGNIYWTDHGFNLIEVARLNGSFRYVIISQGLDQPRSIAVHPEKGLLFWTEWGQMPCIGKARLDGSEKVVLVSMGIAWPNGISIDYEENKLYWCDARTDKIERIDLETGGNREMVLSGSNVDMFSVAVFGAYIYWSDRAHANGSVRRGHKNDATETITMRTGLGVNLKEVKIFNRVREKGTNVCARDNGGCKQLCLYRGNSRRTCACAHGYLAEDGVTCLRHEGYLLYSGRTILKSIHLSDETNLNSPIRPYENPRYFKNVIALAFDYNQRRKGTNRIFYSDAHFGNIQLIKDNWEDRQVIVENVGSVEGLAYHRAWDTLYWTSSTTSSITRHTVDQTRPGAFDREAVITMSEDDHPHVLALDECQNLMFWTNWNEQHPSIMRSTLTGKNAQVVVSTDILTPNGLTIDYRAEKLYFSDGSLGKIERCEYDGSQRHVIVKSGPGTFLSLAVYDNYIFWSDWGRRAILRSNKYTGGDTKILRSDIPHQPMGIIAVANDTNSCELSPCALLNGGCHDLCLLTPNGRVNCSCRGDRILLEDNRCVTKNSSCNAYSEFECGNGECIDYQLTCDGIPHCKDKSDEKLLYCENRSCRRGFKPCYNRRCIPHGKLCDGENDCGDNSDELDCKVSTCATVEFRCADGTCIPRSARCNQNIDCADASDEKNCNNTDCTHFYKLGVKTTGFIRCNSTSLCVLPTWICDGSNDCGDYSDELKCPVQNKHKCEENYFSCPSGRCILNTWICDGQKDCEDGRDEFHCDSSCSWNQFACSAQKCISKHWICDGEDDCGDGLDESDSICGAITCAADMFSCQGSRACVPRHWLCDGERDCPDGSDELSTAGCAPNNTCDENAFMCHNKVCIPKQFVCDHDDDCGDGSDESPQCGYRQCGTEEFSCADGRCLLNTQWQCDGDFDCPDHSDEAPLNPKCKSAEQSCNSSFFMCKNGRCIPSGGLCDNKDDCGDGSDERNCHINECLSKKVSGCSQDCQDLPVSYKCKCWPGFQLKDDGKTCVDIDECSSGFPCSQQCINTYGTYKCLCTDGYEIQPDNPNGCKSLSDEEPFLILADHHEIRKISTDGSNYTLLKQGLNNVIAIDFDYREEFIYWIDSSRPNGSRINRMCLNGSDIKVVHNTAVPNALAVDWIGKNLYWSDTEKRIIEVSKLNGLYPTILVSKRLKFPRDLSLDPQAGYLYWIDCCEYPHIGRVGMDGTNQSVVIETKISRPMALTIDYVNRRLYWADENHIEFSNMDGSHRHKVPNQDIPGVIALTLFEDYIYWTDGKTKSLSRAHKTSGADRLSLIYSWHAITDIQVYHSYRQPDVSKHLCMINNGGCSHLCLLAPGKTHTCACPTNFYLAADNRTCLSNCTASQFRCKTDKCIPFWWKCDTVDDCGDGSDEPDDCPEFRCQPGRFQCGTGLCALPAFICDGENDCGDNSDELNCDTHVCLSGQFKCTKNQKCIPVNLRCNGQDDCGDEEDERDCPENSCSPDYFQCKTTKHCISKLWVCDEDPDCADASDEANCDKKTCGPHEFQCKNNNCIPDHWRCDSQNDCSDNSDEENCKPQTCTLKDFLCANGDCVSSRFWCDGDFDCADGSDERNCETSCSKDQFRCSNGQCIPAKWKCDGHEDCKYGEDEKSCEPASPTCSSREYICASDGCISASLKCNGEYDCADGSDEMDCVTECKEDQFRCKNKAHCIPIRWLCDGIHDCVDGSDEENCERGGNICRADEFLCNNSLCKLHFWVCDGEDDCGDNSDEAPDMCVKFLCPSTRPHRCRNNRICLQSEQMCNGIDECGDNSDEDHCGGKLTYKARPCKKDEFACSNKKCIPMDLQCDRLDDCGDGSDEQGCRIAPTEYTCEDNVNPCGDDAYCNQIKTSVFCRCKPGFQRNMKNRQCEDLNECLVFGTCSHQCINVEGSYKCVCDQNFQERNNTCIAEGSEDQVLYIANDTDILGFIYPFNYSGDHQQISHIEHNSRITGMDVYYQRDMIIWSTQFNPGGIFYKRIHGREKRQANSGLICPEFKRPRDIAVDWVAGNIYWTDHSRMHWFSYYTTHWTSLRYSINVGQLNGPNCTRLLTNMAGEPYAIAVNPKRGMMYWTVVGDHSHIEEAAMDGTLRRILVQKNLQRPTGLAVDYFSERIYWADFELSIIGSVLYDGSNSVVSVSSKQGLLHPHRIDIFEDYIYGAGPKNGVFRVQKFGHGSVEYLALNIDKTKGVLISHRYKQLDLPNPCLDLACEFLCLLNPSGATCVCPEGKYLINGTCNDDSLLDDSCKLTCENGGRCILNEKGDLRCHCWPSYSGERCEVNHCSNYCQNGGTCVPSVLGRPTCSCALGFTGPNCGKTVCEDFCQNGGTCIVTAGNQPYCHCQPEYTGDRCQYYVCHHYCVNSESCTIGDDGSXECVCPTRYEGPKCEVDKCVRCHGGHCIINKDSEDIFCNCTNGKIASSCQLCDGYCYNGGTCQLDPETNVPVCLCSTNWSGTQCERPAPKSSKSDHISTRSIAIIVPLVLLVTLITTLVIGLVLCKRKRRTKTIRRQPIINGGINVEIGNPSYNMYEVDHDHNDGGLLDPGFMIDPTKARYIGGGPSAFKLPHTAPPIYLNSDLKGPLTAGPTNYSNPVYAKLYMDGQNCRNSLGSVDERKELLPKKIEIGIRETVA

SEQ ID NO: 5 - protein, 963 aa, Homo sapiens
MGLPEPGPLRLLALLLLLLLLLLLRLQHLAAAAADPLLGGQGPAKECEKDQFQCRNERCIPSVWRCDEDDDCLDHSDEDDCPKKTCADSDFTCDNGHCIHERWKCDGEEECPDGSDESEATCTKQVCPAEKLSCGPTSHKCVPASWRCDGEKDCEGGADEAGCATLCAPHEFQCGNRSCLAAVFVCDGDDDCGDGSDERGCADPACGPREFRCGGDGGGACIPERWVCDRQFDCEDRSDEAAELCGRPGPGATSAPAACATVSQFACRSGECVHLGWRCDGDRDCKDKSDEADCPLGTCRGDEFQCGDGTCVLAIKHCNQEQDCPDGSDEAGCLQGLNECLHNNGGCSHICTDLKIGFECTCPAGFQLLDQKTCGDIDECKDPDACSQICVNYKGYFKCECYPGYEMDLLTKNCKAAGGKSPSLIFTNRYEVRRIDLVKRNYSRLIPMLKNVVALDVEVATNRIYWCDLSYRKIYSAYMDKASDPKEQEVLIDEQLHSPEGLAVDWVHKHIYWTDSGNKTISVATVDGGRRRTLFSRNLSEPRAIAVDPLRGFMYWSDWGDQAKIEKSGLNGVDRQTLVSDNIEWPNGITLDLLSQRLYWVDSKLHQLSSIDFSGGNRKTLISSTDFLSHPFGIAVFEDKVFWTDLENEAIFSANRLNGLEISILAENLNNPHDIVIFHELKQPRAPDACELSVQPNGGCEYLCLPAPQISSHSPKYTCACPDTMWLGPDMKRCYRAPQSTSTTTLASTMTRTVPATTRAPGTTVHRSTYQNHSTETPSLTAAVPSSVSVPRAPSISPSTLSPATSNHSQHYANEDSKMGSTVTAAVIGIIVPIVVIALLCMSGYLIWRNWKRKNTKSMNFDNPVYRKTTEEEDEDELHIGRTAQIGHVYPAAISSFDRPLWAEPCLGETREPEDPAPALKELFVLPGEPRSQLHQLPKNPLSELPVVKSKRVALSLEDDGLP

SEQ ID NO: 6 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSPEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 7 - protein, 873 aa, Homo sapiens
MGTSALWALWLLLALCWAPRESGATGTGRKAKCEPSQFQCTNGRCITLLWKCDGDEDCVDGSDEKNCVKKTCAESDFVCNNGQCVPSRWKCDGDPDCEDGSDESPEQCHMRTCRIHEISCGAHSTQCIPVSWRCDGENDCDSGEDEENCGNITCSPDEFTCSSGRCISRNFVCNGQDDCSDGSDELDCAPPTCGAHEFQCSTSSCIPISWVCDDDADCSDQSDESLEQCGRQPVIHTKCPASEIQCGSGECIHKKWRCDGDPDCKDGSDEVNCPSRTCRPDQFECEDGSCIHGSRQCNGIRDCVDGSDEVNCKNVNQCLGPGKFKCRSGECIDISKVCNQEQDCRDWSDEPLKECHINECLVNNGGCSHICKDLVIGYECDCAAGFELIDRKTCGDIDECQNPGICSQICINLKGGYKCECSRGYQMDLATGVCKAVGKEPSLIFTNRRDIRKIGLERKEYIQLVEQLRNTVALDADIAAQKLFWADLSQKAIFSASIDDKVGRHVKMIDNVYNPAAIAVDWVYKTIYWTDAASKTISVATLDGTKRKFLFNSDLREPASIAVDPLSGFVYWSDWGEPAKIEKAGMNGFDRRPLVTADIQWPNGITLDLIKSRLYWLDSKLHMLSSVDLNGQDRRIVLKSLEFLAHPLALTIFEDRVYWIDGENEAVYGANKFTGSELATLVNNLNDAQDIIVYHELVQPSGKNWCEEDMENGGCEYLCLPAPQINDHSPKYTCSCPSGYNVEENGRDCQSTATTVTYSETKDTNTTEISATSGLVPGGINVTTAVSEVSVPPKGTSAAWAILPLLLLVMAAVGGYLMWRNWQHKNMKSMNFDNPVYLKTTEEDLSIDIGRHSASVGHTYPAISVVSTDDDLA

SEQ ID NO: 8 - protein, 2887 aa, Homo sapiens
MMKQPQGEQIQINFTHVELQCQSDSSQNYIEVRDGETLLGKVCGNGTISHIKSITNSVWIRFKIDASVEKASFRAVYQVACGDELTGEGVIRSPFFPNVYPGERTCRWTIHQPQSQVILLNFTVFEIGSSAHCETDYVEIGSSSILGSPENKKYCGTDIPSFITSVYNFLYVTFVKSSSTENHGFMAKFSAEDLACGEILTESTGTIQSPGHPNVYPHGINCTWHILVQPNHLIHLMFETFHLEFHYNCTNDYLEVYDTDSETSLGRYCGKSIPPSLTSSGNSLMLVFVTDSDLAYEGFLINYEAISAATACLQDYTDDLGTFTSPNFPNNYPNNWECIYRITVRTGQLIAVHFTNFSLEEAIGNYYTDFLEIRDGGYEKSPLLGIFYGSNLPPTIISHSNKLWLKFKSDQIDTRSGFSAYWDGSSTGCGGNLTTSSGTFISPNYPMPYYHSSECYWWLKSSHGSAFELEFKDFHLEHHPNCTLDYLAVYDGPSSNSHLLTQLCGDEKPPLIRSSGDSMFIKLRTDEGQQGRGFKAEYRQTCENVVIVNQTYGILESIGYPNPYSENQHCNWTIRATTGNTVNYTFLAFDLEHHINCSTDYLELYDGPRQMGRYCGVDLPPPGSTTSSKLQVLLLTDGVGRREKGFQMQWFVYGCGGELSGATGSFSSPGFPNRYPPNKECIWYIRTDPGSSIQLTIHDFDVEYHSRCNFDVLEIYGGPDFHSPRIAQLCTQRSPENPMQVSSTGNELAIRFKTDLSINGRGFNASWQAVTGGCGGIFQAPSGEIHSPNYPSPYRSNTDCSWVIRVDRNHRVLLNFTDFDLESQDSCIMAYDGLSSTMSRLARTCGREQLANPIVSSGNSLFLRFQSGPSRQNRGFRAQFRQACGGHILTSSFDTVSSPRFPANYPNNQNCSWIIQAQPPLNHITLSFTHFELERSTTCARDFVEILDGGHEDAPLRGRYCGTDMPHPITSFSSALTLRFVSDSSISAGGFHTTVTASVSACGGTFYMAEGIFNSPGYPDIYPPNVECVWNIVSSPGNWLQLSFISFQLEDSQDCSRDFVEIREGNATGHLVGRYCGNSFPLNYSSIVGHTLWVRFISDGSGSGTGFQATFMKIFGNDNIVGTHGKVASPFWPENYPHNSNYQWTVNVNASHVVHGRILEMDIEEIQNCYYDKLRIYDGPSIHARLIGAYCGTQTESFSSTGNSLTFHFYSDSSISGKGFLLEWFAVDAPDGVLPTIAPGACGGFLRTGDAPVFLFSPGWPDSYSNRVDCTWLIQAPDSTVELNILSLDIESHRTCAYDSLVIRDGDNNLAQQLAVLCGREIPGPIRSTGEYMFIRFTSDSSVTRAGFNASFHKSCGGYLHADRGIITSPKYPETYPSNLNCSWHVLVQSGLTIAVHFEQPFQIPNGDSSCNQGDYLVLRNGPDIYSPPLGPPGGNGHFCGSHASSTLFTSDNQMFVQFISDHSNEGQGFKIKYEAKSLACGGNVYIHDADSAGYVTSPNHPHNYPPHADCIWILAAPPETRIQLQFEDRFDIEVTPNCTSNYLELRDGVDSDAPILSKFCGTSLPSSQWSSGEVMYLRFRSDNSPTHVGFKAKYSIAQCGGRVPGQSGVVESIGHPTLPYRDNLFCEWHLQGLSGHYLTISFEDFNLQNSSGCEKDFVEIWDNHTSGNILGRYCGNTIPDSIDTSSNTAVVRFVTDGSVTASGFRLRFESSMEECGGDLQGSIGTFTSPNYPNPNPHGRICEWRITAPEGRRITLMFNNLRLATHPSCNNEHVIVFNGIRSNSPQLEKLCSSVNVSNEIKSSGNTMKVIFFTDGSRPYGGFTASYTSSEDAVCGGSLPNTPEGNFTSPGYDGVRNYSRNLNCEWTLSNPNQGNSSISIHFEDFYLESHQDCQFDVLEFRVGDADGPLMWRLCGPSKPTLPLVIPYSQVWIHFVTNERVEHIGFHAKYSFTDCGGIQIGDSGVITSPNYPNAYDSLTHCSSLLEAPQGHTITLTFSDFDIEPHTTCAWDSVTVRNGGSPESPIIGQYCGNSNPRTIQSGSNQLVVTFNSDHSLQGGGFYATWNTQTLGCGGIFHSDNGTIRSPHWPQNFPENSRCSWTAITHKSKHLEISFDNNFLIPSGDGQCQNSFVKVWAGTEEVDKALLATGCGNVAPGPVITPSNTFTAVFQSQEAPAQGFSASFVSRCGSNFTGPSGYIISPNYPKQYDNNMNCTYVIEANPLSVVLLTFVSFHLEARSAVTGSCVNDGVHIIRGYSVMSTPFATVCGDEMPAPLTIAGPVLLNFYSNEQITDFGFKFSYRIISCGGVFNFSSGIITSPAYSYADYPNDMHCLYTITVSDDKVIELKFSDFDVVPSTSCSHDYLAIYDGANTSDPLLGKFCGSKRPPNVKSSNNSMLLVFKTDSFQTAKGWKMSFRQTLGPQQGCGGYLTGSNNTFASPDSDSNGMYDKNLNCVWIIIAPVNKVIHLTFNTFALEAASTRQRCLYDYVKLYDGDSENANLAGTFCGSTVPAPFISSGNFLTVQFISDLTLEREGFNATYTIMDMPCGGTYNATWTPQNISSPNSSDPDVPFSICTWVIDSPPHQQVKITVWALQLTSQDCTQNYLQLQDSPQGHGNSRFQFCGRNASAVPVFYSSMSTAMVIFKSGVVNRNSRMSFTYQIADCNRDYHKAFGNLRSPGWPDNYDNDKDCTVTLTAPQNHTISLFFHSLGIENSVECRNDFLEVRNGSNSNSPLLGKYCGTLLPNPVFSQNNELYLRFKSDSVTSDRGYEIIWTSSPSGCGGTLYGDRGSFTSPGYPGTYPNNTYCEWVLVAPAGRLVTINFYFISIDDPGDCVQNYLTLYDGPNASSPSSGPYCGGDTSIAPFVASSNQVFIKFHADYARRPSAFRLTWDS